(19) United States
(12) Patent Application Publication
(10) Pub. No.: US 2023/0053227 A1
(43) Pub. Date: Feb. 16, 2023

(54) DIETARY FIBER COMPOSITIONS

(71) Applicant: G&S Laboratories, Inc., Eagan, MN (US)

(72) Inventors: Robert A. Ganz, Minnetonka, MN (US); Mo E. Saremi, Eagan, MN (US); William Christopfel, Edina, MN (US)

(21) Appl. No.: 17/874,994

(22) Filed: Jul. 27, 2022

Related U.S. Application Data
(60) Provisional application No. 63/345,562, filed on May 25, 2022.
(60) Provisional application No. 63/305,076, filed on Jan. 31, 2022.
(60) Provisional application No. 63/236,849, filed on Aug. 25, 2021.
(60) Provisional application No. 63/203,599, filed on Jul. 27, 2021.

(51) Int. Cl. A61K 36/68; A23L 33/22; A23L 33/24; A61K 31/733; A61K 31/718; A61K 31/716; A61K 31/702; A61P 29/00; A61P 1/00; A61P 3/10

(52) U.S. Cl. CPC A61K 36/68; A23L 33/22; A23L 33/24; A61K 31/733; A61K 31/718; A61K 31/716; A61K 31/702; A61P 29/00; A61P 1/00; A61P 3/10; A23V 2002/00

(57) ABSTRACT

The present disclosure relates to compositions comprising dietary fibers. For example, in some embodiments, the composition comprises two or more fibers (e.g., fibers such as beta-glucan, wheat dextrin, inulin, pectin, psyllium, or potato starch) each present in amount of about 40% to about 60% w/w of the composition. In some embodiments, the composition further includes a sweetener and/or a flavoring agent. Such compositions are useful in the treatment or improvement of gastrointestinal diseases.

### CROSS-REFERENCE TO RELATED APPLICATIONS

This application claims priority to U.S. Provisional Application No. 63/345,562, filed May 25, 2022; U.S. Provisional Application No. 63/305,076, filed Jan. 31, 2022; U.S. Provisional Application No. 63/236,849, filed Aug. 25, 2021; and U.S. Provisional Application No. 63/203,599, filed Jul. 27, 2021; each of these prior applications is incorporated herein by reference in its entirety.

### TECHNICAL FIELD

The present disclosure relates to compositions comprising dietary fibers. Such fibers are useful for treating or improving constipation, hemorrhoids, anal fissures, diarrhea, fecal incontinence, irritable bowel syndrome, inflammatory bowel disease (Crohn's disease and ulcerative colitis), microscopic colitis, leaky gut, certain metabolic syndromes such as glucose intolerance, prediabetes, diabetes, nonalcoholic fatty liver disease and obesity, and for altering the human microbiome in a favorable manner.

### BACKGROUND

Dietary fibers are a very important part of the human diet for a variety of reasons. For example, a diet high in insoluble dietary fiber (e.g., wheat bran) is recommended for individuals who require bulking of the stool, such as those with constipation, hemorrhoids, anal fissures, diarrhea, fecal incontinence and some types of irritable bowel syndrome, and a diet high in soluble or partially soluble fiber (e.g., psyllium, pectin, wheat dextrin, and oat products) can improve the glycogenic response to diet, which can improve blood glucose levels and some types of irritable bowel syndrome and can reduce the risk for type 2 diabetes, atherosclerosis, and stroke. Fiber intake may also be important with regard to systemic inflammation and/or leaky gut syndrome through, for example, the commensal relationship between the human gut and the bacteria (microbiome) within it. Bacteria in humans perform some functions that human cells cannot, such as generating some B vitamins, generating vitamin K, extracting nutrients from indigestible fiber, and generating short chain fatty acids (SCFAs). Importantly, SCFAs are derived from dietary fibers and are a source of energy for epithelial colonocytes. The types of SCFAs derived from the dietary fiber(s) depends on the dietary fiber, and the types of SCFAs have an impact on the health of colonocytes. An adequate amount of fiber is essential to maintain healthy gut ecology. Methods and compositions for improving, for example, one or more of: irritable bowel syndrome, glycogenic response to diet, gut ecology, and bulking of the stool are highly desirable.

### SUMMARY

Provided herein are compositions including a first fiber present in an amount of about 20% to about 80% w/w of the composition and a second fiber present in an amount of about 20% to about 80% w/w of the composition.

In some embodiments, the first fiber is present in an amount of about 35% to about 65% w/w of the composition. In some embodiments, the first fiber is present in an amount of about 45% to about 55% w/w of the composition. In some embodiments, the first fiber is present in an amount of about 50% w/w of the composition.

In some embodiments, the second fiber is present in an amount of about 35% to about 65% w/w of the composition. In some embodiments, the second fiber is present in an amount of about 45% to about 55% w/w of the composition. In some embodiments, the second fiber is present in an amount of about 50% w/w of the composition.

In some embodiments, the first fiber is present in an amount of about 1 grams to about 6 grams. In some embodiments, the first fiber is present in an amount of about 2 grams to about 4 grams. In some embodiments, the second fiber is present in an amount of about 1 grams to about 6 grams. In some embodiments, the second fiber is present in an amount of about 2 grams to about 4 grams.

In some embodiments, the ratio of the first fiber to the second fiber is about 2:3 to about 3:2. In some embodiments, the ratio of the first fiber to the second fiber is about 1:1.

In some embodiments, the first fiber is selected from the group consisting of: psyllium, pectin, guar, a lignin, cellulose, hemicellulose, resistant starch, a resistant dextrin, inulin, a chitin, pectin, a beta-glucan, potato starch, cornstarch, and an oligosaccharide.

In some embodiments, the second fiber is selected from the group consisting of: psyllium, pectin, guar, a lignin, cellulose, hemicellulose, a resistant starch, a resistant dextrin, inulin, a chitin, pectin, a beta-glucan, potato starch, cornstarch and an oligosaccharide, wherein the second fiber is different from the first fiber.

In some embodiments, the first fiber is psyllium. In some embodiments, the second fiber is selected from the group consisting of: wheat dextrin, inulin, potato starch, pectin, cornstarch and an oligosaccharide. In some embodiments, the second fiber is wheat dextrin. In some embodiments, the second fiber is inulin. In some embodiments, the second fiber is potato starch.

In some embodiments, the first fiber is wheat dextrin. In some embodiments, the second fiber is selected from the group consisting of: psyllium, inulin, potato starch, pectin, cornstarch and an oligosaccharide. In some embodiments, the second fiber is inulin. In some embodiments, the second fiber is potato starch.

In some embodiments, the first fiber is beta-glucan. In some embodiments, the second fiber is selected from the group consisting of: psyllium, inulin, potato starch, pectin, cornstarch, wheat dextrin, and an oligosaccharide. In some embodiments, the second fiber is psyllium. In some embodiments, the second fiber is wheat dextrin.

In some embodiments, the composition further includes a third fiber. In some embodiments, the third fiber is an oligosaccharide. In some embodiments, the oligosaccharide is selected from the group consisting of: fructooligosaccharide (FOS), maltose, iso-maltose, a maltosyl-iso-malto-oligosaccharide, and a combination thereof. In some embodiments, the oligosaccharide is FOS. In some embodiments, the third fiber is present in an amount of about 1% to about 10% w/w of the composition.

Also provided herein are compositions including psyllium present in an amount of about 45% to about 55% w/w of the composition and wheat dextrin present in an amount of about 45% to about 55% w/w of the composition. Also provided herein are compositions including psyllium present in an amount of 45% to about 55% w/w of the composition and inulin present in an amount of 45% to about 55% w/w of the composition. Also provided herein are compositions including wheat dextrin present in an amount of 45% to about 55% w/w of the composition and inulin present in an amount of 45% to about 55% w/w of the composition.

In some of any of the above embodiments, the psyllium is present in an amount of about 50% w/w of the composition. In some of any of the above embodiments, the wheat dextrin is present in an amount of about 50% w/w of the composition. In some of any of the above embodiments, the inulin is present in an amount of about 50% w/w of the composition.

In some of any of the above embodiments, the composition further includes an oligosaccharide. In some embodiments, the oligosaccharide is selected from the group consisting of: fructooligosaccharide (FOS), maltose, iso-maltose, a maltosyl-iso-malto-oligosaccharide, and a combination thereof. In some embodiments, the oligosaccharide is FOS. In some embodiments, the oligosaccharide is present in an amount of about 1% to about 10% w/w of the composition.

In some of any of the above embodiments, the composition further includes a preservative. In some embodiments, the preservative includes: a palmitate, a benzoate, benzoic acid, a propionate, a sorbate, sorbic acid, an erythorbate, a nitrite, ethylenediaminetetraacetic acid (EDTA), butylated hydroxyanisole (BHA), butylated hydroxytoluene (BHT), capryllic acid, dilauryl thiodipropionate, erythorbic acid, gum guaiac, methylparaben, a sulfite, a bisulfite, a metabisulfite, propyl gallatepy, propylparaben, stannous chloride, sulfur dioxide, thiodipropionic acid, a tocopherol, or a combination thereof.

In some of any of the above embodiments, the composition further includes a sweetener. In some embodiments, the sweetener includes an intense sweetener, a natural sugar, or a combination thereof. In some embodiments, the sweetener includes sorbitol, sucrose, a saccharin, a cyclamate, aspartame, sucralose, thaumatin, acesulfam K, xylose, ribose, glucose, mannose, galactose, fructose, dextrose, sucrose, maltose, starch or corn syrup, sorbitol, xylitol, mannitol, glycerin, or a combination thereof.

In some of any of the above embodiments, the composition further includes a flavoring agent. In some embodiments, the flavoring agent includes a natural flavoring agent, a natural fruit flavoring agent, an artificial flavoring agent, an artificial fruit flavoring agent, a flavor enhancer, or a combination thereof.

In some of any of the above embodiments, the composition is a solid composition. In some of any of the above embodiments, the composition is a powder. In some of any of the above embodiments, the composition is homogenous.

Also provided herein are methods for reducing inflammation in a subject in need thereof comprising administering to the subject any of the compositions described herein. In some embodiments, provided herein are methods for improving intestinal health in a subject in need thereof comprising administering to the subject any of the compositions described herein. Also provided herein are methods for improving a gastrointestinal disease in a subject in need thereof comprising administering to the subject any of the compositions described herein.

In some embodiments, provided herein are methods for improving diabetes or prediabetes in a subject in need thereof comprising administering to the subject any of the compositions described herein. Also provided herein are methods for improving blood glucose levels in a subject in need thereof comprising administering to the subject any of the compositions described herein.

In some of any of the above embodiments, a method provided herein includes increasing the diversity of bacteria in the gastrointestinal tract of the subject.

Also provided herein are methods for increasing the diversity of bacteria in the gastrointestinal tract of a subject in need thereof, the method comprising administering to the subject any of the compositions described herein.

In some of any of the above embodiments, the method includes determining the bacterial composition in a sample from the subject.

In some of any of the above embodiments, the method includes restoring the level of one or more bacterial metabolites selected from lactate, an SCFA, a bile acid, a branched chain amino acid, tryptophan, trimethylamine N-oxide (TMAO), and an indole derivative in the subject.

Also provided herein are methods for restoring the level of one or more bacterial metabolites in a subject in need thereof, the method comprising administering any of the compositions described herein. In some embodiments, the level of one or more bacterial metabolites selected from: lactate, SCFA, bile acid, branched chain amino acid, tryptophan, TMAO, and an indole derivative is restored in the gastrointestinal tract of the subject.

In some embodiments, the method includes detecting the level of one or more of: lactate, an SCFA, a bile acid, a branched chain amino acid, tryptophan, TMAO, and an indole derivative in a sample from the subject. In some embodiments, the level of the lactate, SCFA, bile acid, branched chain amino acid, tryptophan, TMAO, or indole derivative in the sample is restored in comparison to the same bacterial metabolite in a reference sample (e.g., a sample from a control individual or an average of the level of the bacterial metabolite from samples from a control population).

In some embodiments, the sample is obtained after administration of any of the compositions described herein. In some embodiments, the reference sample is a sample from the subject obtained prior to the administration of any of the compositions described herein. In some embodiments, the sample is a fecal sample. In some embodiments, the reference sample is a fecal sample.

Also provided herein are methods for improving intestinal health in a subject in need thereof, the method comprising: (a) determining that a first sample from the subject has an increased level of one or more bacterial metabolites selected from: lactate, an SCFA, a bile acid, a branched chain amino acid, tryptophan, TMAO, and an indole derivative compared to the level of the same bacterial metabolite in a reference sample; and (b) administering any of the compositions described herein.

In some embodiments, the gastrointestinal tract of the subject has an increased level of the one or more bacterial metabolites. In some embodiments, the first sample from the subject has an increased the level of one or more bacterial metabolites selected from: lactate, an SCFA, a bile acid, a branched chain amino acid, tryptophan, TMAO, and an indole derivative compared to a reference sample. In some embodiments, the level of one or more bacterial metabolites selected from: lactate, SCFA, bile acid, branched chain amino acid, tryptophan, TMAO, and indole derivative is decreased in a second sample from the subject compared to the level of the same bacterial metabolite in the first sample.

Also provided herein are methods for improving intestinal health in a subject in need thereof, the method comprising: (a) determining that a sample from the subject has an decreased level of one or more bacterial metabolites selected from: lactate, an SCFA, a bile acid, a branched chain amino acid, tryptophan, TMAO, and an indole derivative compared to the level of the same bacterial metabolite in a reference sample; and (b) administering any of the compositions described herein. In some embodiments, the gastrointestinal tract of the subject has a decreased level of the one or more bacterial metabolites. In some embodiments, the first sample from the subject has decreased level of the one or more bacterial metabolites selected from: lactate, an SCFA, a bile acid, a branched chain amino acid, tryptophan, TMAO, and an indole derivative compared to a reference sample. In some embodiments, the level of one or more bacterial metabolites selected from: lactate, SCFA, bile acid, branched chain amino acid, tryptophan, TMAO, and indole derivative is increased in a second sample from the subject compared to the level of the same bacterial metabolite in the first sample.

In some embodiments, the method comprises detecting the level of one or more bacterial metabolites selected from lactate, an SCFA, a bile acid, a branched chain amino acid, tryptophan, TMAO, and an indole derivative in a first sample from the subject.

In some embodiments, the method comprises detecting the level of one or more bacterial metabolites selected from lactate, an SCFA, a bile acid, a branched chain amino acid, tryptophan, TMAO, and an indole derivative in a second sample from the subject.

In some embodiments, the second sample is obtained after administration of any of the compositions described herein.

In some embodiments, the first sample is a fecal sample. In some embodiments, the second sample is a fecal sample.

In some of any of the above embodiments, the ratio of SCFAs in the sample from the subject is about 78:12:10 to about 60:20:20 acetate:propionate:butyrate after administration of any of the compositions described herein. In some embodiments, the ratio of SCFAs in the sample from the subject is about 78:12:10 acetate:propionate:butyrate after administration of the composition of any of the compositions described herein. In some embodiments, the ratio of SCFAs in the sample from the subject is about 60:20:20 acetate:propionate:butyrate after administration of any of the compositions described herein.

In some of any of the above embodiments, the level of TMAO in the sample from the subject is about 0.8 to about 2.2 μmol/L after administration of any of the compositions described herein. In some embodiments, the level of TMAO in the sample from the subject is about 1.5 μmol/L after administration of any of the compositions described herein.

In some of any of the above embodiments, the level of the indole derivative in the sample from the subject is about 15-25 nM after administration of the composition of any of the compositions described herein. In some embodiments, the level of the indole derivative in the sample from the subject is about 20 nM after administration of the composition of any of the compositions described herein. In some embodiments, the indole derivative is imidazole propionate.

In some of any of the above embodiments, the level of the lactate in the sample from the subject is about 1 to about 6 mmol/L after administration of any of the compositions described herein. In some embodiments, the level of the lactate in the sample from the subject is 6 mmol/L or less after administration of any of the compositions described herein.

In some of any of the above embodiments, the subject has a gastrointestinal disease selected from the group consisting of: a foregut disease, flatulence, intestinal gas, bloating, leaky gut syndrome, constipation, hemorrhoid, anal fissure, diarrhea, fecal incontinence, irritable bowel syndrome, inflammatory bowel disease, microscopic colitis, small intestinal bacterial overgrowth, glucose intolerance, prediabetes, diabetes, coronary artery disease, nonalcoholic fatty liver disease, steatohepatitis, and obesity. In some embodiments, the foregut disease is selected from the group consisting of: GERD, Barrett's esophagus, esophageal cancer, esophageal motility disorders, eosinophilic esophagitis, achalasia, oral cancer, halitosis, achalasia, esophageal diverticula, and periodontal gum disease.

As used herein, the phrases an “effective amount” or a “therapeutically effective amount” of an active agent or ingredient, or pharmaceutically active agent or ingredient, refer to an amount of the active agent or pharmaceutically active agent sufficient enough to reduce or eliminate one or more symptoms of the disorder or to effect a cure upon administration. Effective amounts of the active agent or pharmaceutically active agent will vary with the kind of active agent or pharmaceutically active agent chosen, the particular condition or conditions being improved or treated, the severity of the condition, the duration of the treatment, the specific components of the composition being used, and like factors.

As used herein, “subject” or “patient” refers to any subject, particularly a mammalian subject, for whom diagnosis, prognosis, or therapy is desired, for example, a human.

As used herein, a “treatment” or “treating” of a disease, disorder, or condition encompasses alleviation of at least one symptom thereof, a reduction in the severity thereof, or the delay or inhibition of the progression thereof. Treatment need not mean that the disease, disorder, or condition is totally cured. A useful composition herein needs only to reduce the severity of a disease, disorder, or condition, reduce the severity of one or more symptoms associated therewith, or provide improvement to a patient or subject's quality of life.

As used herein “improvement” of or “improving” a disease, disorder, or condition means reducing the severity of a disease, disorder, or condition, reducing the severity of one or more symptoms associated therewith, or increasing a patient or subject's quality of life.

As used herein “restoration” of or “restoring” a level of a bacterial metabolite means altering the level of the bacterial metabolite in a subject or a sample from a subject to a level similar to or the same as the level of the same metabolite in a control sample, control individual, or control population. The level of a bacterial metabolite from a control population can be a level determined by averaging the amount of the bacterial metabolite in a sample from each individual in the control population.

A control population can include individuals that meet one or more qualifications. Such qualifications can include, for example, that the individuals have not been diagnosed with a disease (e.g., the same disease as the subject); the individuals do not have a known genetic predisposition to a disease (e.g., the same disease as the subject); the individuals do not have a known environmental predisposition to a disease (e.g., the same disease as the subject); or the individuals do not have a known genetic or environmental predisposition that would prevent treatment of and/or recovery from a disease (e.g., the same disease as the subject). In some embodiments, the individuals in the control population meet one, two, three, or more of the above control population qualifications. In some embodiments, the control population is homogenous with respect to at least one of the qualifications.

As used herein, unless otherwise noted, the term “prevention” shall include (a) reduction in the frequency of one or more symptoms; (b) reduction in the severity of one or more symptoms; (c) the delay or avoidance of the development of additional symptoms; and/or (d) delay or avoidance of the development of the disorder or condition.

Reference to the term “about” has its usual meaning in the context of pharmaceutical compositions to allow for reasonable variations in amounts that can achieve the same effect and also refers herein to a value of plus or minus 10% of the provided value. For example, “about 20” means or includes amounts from 18 to and including 22.

As used herein, “fibers” are carbohydrates mostly derived from plants that are resistant to digestion by human enzymes. In some embodiments, a fiber is a polysaccharide.

The terms “partially hydrolyzed fibers” or “a partially hydrolyzed fiber” refers to fibers that have hydrolysable chemical bonds that have been hydrolyzed, but where not all of the hydrolyzable chemical bonds have been hydrolyzed. For example, wherein from about 1% to about 99% of the hydrolysable chemical bonds have been hydrolyzed. For example, about 1%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 99% of the hydrolysable chemical bonds have been hydrolyzed. In some embodiments, about 10% to about 99% of the hydrolysable chemical bonds have been hydrolyzed. The extent of hydrolysis of a polysaccharide (e.g., a fiber) can be determined by any of the methods known to one of skill in the art. For example, the number of reducing ends of polysaccharides (e.g., fibers) can be determined and that can be used to calculate the number of sugars per chain to determine the length of the polysaccharide (e.g., the fiber). In some embodiments, these methods can be colorimetric and/or based on the ability of aldehydes to reduce other molecules. As another example, the molecular weight of a polymer, such as a polysaccharide, fiber, or partially hydrolyzed fiber, can be determined by a variety of techniques. Non-limiting examples of techniques to determine the molecular weight of a polymer include gel permeation chromatography (GPC), static light scattering (SLS), and intrinsic viscosity (IV). In some cases, certain types of fibers can partially hydrolyze when added to water.

The chain length of a partially hydrolyzed polysaccharide (e.g., a partially hydrolyzed fiber) can be related to the viscosity of the composition comprising the partially hydrolyzed polysaccharide (e.g., a partially hydrolyzed fiber). In some embodiments, the extent of hydrolysis of the partially hydrolyzed fiber is quantified by the viscosity of the composition comprising the partially hydrolyzed fiber. The viscosity of a solution comprising one or more fibers can depend on the extent of the hydrolysis of the one or more fibers. For example, the viscosity of a solution comprising one or more partially hydrolyzed fibers can decrease with increasing hydrolysis of one or more of the fibers. In some embodiments, the greater the extent of hydrolysis of the partially hydrolyzed fiber (e.g., the higher the percentage of hydrolyzed chemical bonds), the greater the decrease in the viscosity of a solution comprising the partially hydrolyzed fiber compared to a solution comprising the unhydrolyzed fiber.

“Room temperature” or “RT” refers to the ambient temperature of a typical laboratory, which is typically around 25° C.

The term “dietary fiber combination” as used herein means a product that results from the mixing or combining of more than one dietary fiber and includes both fixed and non-fixed combinations of the dietary fibers. The term “fixed combination” means that the dietary fibers, e.g. a first fiber and a second fiber, are both administered to a subject simultaneously in the form of a single entity or dosage. The term “non-fixed combination” means that the dietary fibers, e.g. a first fiber and a second fiber, are both administered to a patient as separate entities either simultaneously, concurrently or sequentially with no specific time limits, wherein such administration provides therapeutically effective levels of the two dietary fibers in the body of the patient. The latter also applies to the administration of three or more dietary fibers.

As used herein, “microbiome” refers to the collection of microbes from a given environment and/or the genetic material from the collection of microbes from a given environment. For example, “microbiome” can refer to the collection of microbes (e.g., bacteria, archea, fungi, protozoa, and viruses) from the gastrointestinal tract of a human and/or the genetic material from the collection of microbes from the gastrointestinal tract of a human.

As used herein, “metabolome” refers to the collection of small molecules from a given environment. For example, “metabolome” can refer to the collection of microbial products or metabolites from a given environment (e.g., the gastrointestinal tract of a human).

The details of one or more embodiments of the invention are set forth in the accompanying the description below. Other features, objects, and advantages of the invention will be apparent from the description and from the claims.

### DETAILED DESCRIPTION

Dietary fibers (also referred to herein as “fibers”) include non-starch polysaccharides and other plant components such as cellulose, hemicellulose, resistant starch, resistant dextrins, inulin, lignins, chitins, pectins, guar, beta-glucans, and oligosaccharides. Fiber is mostly derived from plants, and it consists of the edible portion of a plant-based diet that cannot be digested by the human digestive system.

Fiber is typically divided into soluble fiber, i.e., fiber that can dissolve in water, and insoluble fiber, which cannot dissolve in water. Insoluble fiber comes from the cell walls of plants and this type of fiber does not dissolve in water. Examples of foods that contain insoluble fiber include potato starch, wheat, rye and other grains such as bran. Some types of insoluble fibers contain complex carbohydrates such as resistant starches and can be fermented by bacteria. Thus, some types of insoluble fiber can also act as a prebiotic and produce short chain fatty acids (SCFAs), which are physiologically active, but other types of insoluble fibers, such as lignins, cannot be fermented, and they largely act as bulking agents. Insoluble dietary fibers can help bulk stool, which is recommended for individuals with constipation, hemorrhoids, anal fissures, diarrhea, fecal incontinence and some types of irritable bowel syndrome.

Soluble fiber is composed of carbohydrates, and certain types can be readily fermented by gut bacteria in the small bowel or colon. Soluble, or partially soluble, dietary fibers, such as psyllium, pectin, wheat dextrin, inulin, and beta-glucan (e.g., from oat products), can improve the glycogenic response to diet. Non-limiting examples of foods that contain soluble fiber include fruits, oats, barley, and legumes such as peas and corn. Non-limiting examples of soluble fibers include psyllium, pectin, wheat dextrin, inulin, oligosaccharides, and beta-glucan (e.g., from oat products). Oligosaccharides can include fructooligosaccharides (FOS) also known as oligofructose, maltose, iso-maltose, maltosyl-iso-malto-oligosaccharides, galactooligosaccharides, and mannan-oligosaccharides (MOS). FOS are composed of linear chains of fructose units that are linked by beta (2-1) bonds. The number of fructose units ranges from 2 to 60 and often terminate in a glucose unit. Inulin and FOS can be derived from chicory root fiber. In some embodiments, FOS is degraded (e.g., hydrolyzed) inulin. In some embodiments, FOS is synthesized enzymatically (see, e.g., Lorenzoni et al. Carbohydr. Polym. 2014; 103:193-7). Commercial psyllium is composed mainly of arabinose (22%), xylose (57%), and uronic acids (10-15%), with small amounts of galactose, rhamnose, glucose, and mannose; it is a highly branched acidic arabinoxylan with a high molecular weight. Psyllium is derived from the seeds of the plant genus Plantago, including Plantago ovata, an herb mainly grown in India.

Fiber intake can also play an important role regarding the relationship between the human gut and the bacteria within it. Humanoids have co-evolved with bacteria for over 1 million years, and humans have co-evolved with bacteria for about the last 300,000 years. It is estimated that humans contain over 1 trillion bacterial cells, mostly on the skin and in the gut; most of the bacteria found in the gut are in the colon.

To keep the bacteria from causing inflammation in the colon wall, humans have evolved four separate defenses: 1) the bacterial ecology is organized such that humans are mainly populated by non-inflammatory species that keep pathogens (e.g., Clostridium difficile) and pro-inflammatory commensals in check; 2) humans have a thick mucus layer that contains viral phages that destroy bacteria; 3) below the mucus layer is the epithelium with tight junctions; and 4) below that is the submucosa with plasma cells that secrete IgA, lymphocytes, antimicrobial peptides (AMPs), etc.

Some bacteria have co-evolved with humans as commensals, for example, bacteria in humans perform some functions that human cells cannot, such as generating some B vitamins, generating vitamin K, extracting nutrients from indigestible fiber, and generating short chain fatty acids (SCFAs). Certain SFCAs such as propionic acid, butyric acid and acetic acid are necessary for colon health, because they are the sole nutritional source for colonocytes; without SCFAs, the lining of the colon would atrophy and potentially lead to the development of colitis. There is a very important interplay between SCFAs, gut microbiota, and host energy metabolism. See, e.g., Agus et al. Gut. 2021; 70(6):1174-1182, which is incorporated by reference herein in its entirety. For example, soluble fiber typically becomes viscous in the human gastrointestinal tract and certain types of fiber can drive the growth of certain gut bacterial species. While some fibers, for example, psyllium, are not particularly fermentable and so, they do not generate many SCFAs, hydrolysis of psyllium generates shorter chain oligosaccharides, e.g., various monosaccharides, disaccharides, trisaccharides, etc. leading to carbohydrate monomers and polymers of variable length, which are more fermentable.

Humans are largely populated by four bacterial families: bacteroides, firmacutes, proteobacters, and acintobacters. While developed and undeveloped human populations have very different distributions of these families, and their respective genuses and species, across all human populations there are consistently 15-17 key foundational species within these groups that sustain a healthy gut ecology. However, if these foundational species are suppressed (e.g., by antibiotics or a diet high in fat and sugar and low in fiber), other commensals may grow more populous, and the gut can lose foundational species. The other new commensals may have a more inflammatory interaction with colonocytes, may not generate the same amount of SCFAs, and/or may generate the wrong type of SCFAs. This may lead to an inflammatory interaction or atrophy of colonocytes, which may open up the tight cell junctions potentially causing a leaky gut, thin out or change the mucus lining, or cause an increase in submucosal white cell populations.

Dietary fiber intake may also have a role in systemic inflammation and leaky gut syndrome, e.g., a breach in the mucosal barrier of the intestine that may allow bacteria, bacterial antigens, and bacterial lipopolysaccharide (LPS) to enter the blood stream, which may cause generalized or local inflammation and possibly autoimmune diseases. For example, if the bacteria/LPS products get into the liver, in the presence of obesity, humans can get non-alcoholic fatty liver disease or nonalcoholic steatohepatitis (NAFLD/NASH) and cirrhosis; in the pancreas and periphery, humans can get insulin resistance; in the colon, humans can get irritable bowel syndrome; and in the coronary arteries, humans can get atherosclerosis. Other diseases that may be linked to leaky gut include CAD/MI, clotting disorders, and celiac sprue and inflammatory bowel diseases (e.g., Crohn's and ulcerative colitis), which are consistently associated with an increased risk in many autoimmune diseases. Hence, dietary fibers, and the types of fibers ingested, play an important, multifaceted and complex role in human health and disease. The compositions provided herein can be useful for increasing the diversity of the microbiome in the gastrointestinal tract of a subject. The compositions provided herein can also restore the levels of bacterial metabolites, such as SCFAs, in subjects that have an increased or decreased level of the bacterial metabolites compared to a control population.

### Dietary Fiber Compositions

Provided herein are compositions including two or more fibers (e.g., a first fiber, a second fiber, a third fiber, a fourth fiber, and a fifth fiber). Non-limiting examples of a fiber include non-starch polysaccharides and other plant components such as cellulose, hemicellulose, resistant starch, resistant dextrins, inulin, lignins, chitins, pectins, guar, beta-glucans, potato starch and oligosaccharides. In some embodiments, the composition has two fibers (e.g., a first fiber and a second fiber). In some embodiments, the composition has three fibers (e.g., at least a first fiber, a second fiber, and a third fiber). In some embodiments, the composition has four fibers (e.g., a first fiber, a second fiber, a third fiber, and a fourth fiber). In some embodiments, the composition has five fibers (e.g., a first fiber, a second fiber, a third fiber, a fourth fiber, and a fifth fiber).

In some embodiments, the two or more fibers (e.g., the two, three, four, or five fibers) are together present in an amount of about 50% to about 100% w/w of the composition. For example, the two or more fibers are together present in an amount of about 50% to about 60%, about 50% to about 70%, about 50% to about 80%, about 50% to about 90%, about 60% to about 70%, about 60% to about 80%, about 60% to about 90%, about 60% to about 100%, about 70% to about 80%, about 70% to about 90%, about 70% to about 100%, about 80% to about 90%, about 80% to about 100%, or about 90% to about 100% w/w of the composition. In some embodiments, the two or more fibers are together present in an amount of about 80%, about 85%, about 80%, about 90%, about 91%, about 92%, about 93%, about 94%, about 95%, about 96%, about 97%, about 98%, about 99%, or about 100% w/w of the composition.

In some embodiments, each of the fibers (e.g., each of the first fiber, the second fiber, the third fiber, the fourth fiber, or the fifth fiber) is independently present in an amount of about 1% to about 80% w/w of the composition. For example, each of the fibers (e.g., each of the first fiber, the second fiber, the third fiber, the fourth fiber, or the fifth fiber) is independently present in an amount of about 1% to about 70%, about 1% to about 60%, about 1% to about 50%, about 1% to about 40%, about 1% to about 30%, about 1% to about 20%, about 1% to about 10%, about 1% to about 5%, about 5% to about 80%, about 5% to about 70%, about 5% to about 60%, about 5% to about 50%, about 5% to about 40%, about 5% to about 30%, about 5% to about 20%, about 5% to about 10%, about 10% to about 80%, about 10% to about 70%, about 10% to about 60%, about 10% to about 50%, about 10% to about 40%, about 10% to about 30%, about 10% to about 20%, about 20% to about 70%, about 20% to about 60%, about 20% to about 50%, about 20% to about 40%, about 20% to about 30%, about 30% to about 80%, about 30% to about 70%, about 30% to about 60%, about 30% to about 50%, about 30% to about 40%, about 40% to about 80%, about 40% to about 70%, about 40% to about 60%, about 40% to about 50%, about 50% to about 80%, about 50% to about 70%, about 50% to about 60%, about 60% to about 80%, about 60% to about 70%, or about 70% to about 80% w/w of the composition. In some embodiments, each of the fibers (e.g., each of the first fiber, the second fiber, the third fiber, the fourth fiber, or the fifth fiber) is independently present in an amount of about 35% to about 45%, about 45% to about 55%, about 35% to about 40%, about 40% to about 45%, about 45% to about 50%, about 50% to about 55%, about 55% to about 60%, or about 60% to about 65% w/w of the composition. In some embodiments, each of the fibers (e.g., each of the first fiber, the second fiber, the third fiber, the fourth fiber, or the fifth fiber) is independently present in an amount of about 2% to about 8%, about 5% to about 15%, or about 15% to about 25% w/w of the composition. In some embodiments, each of the fibers (e.g., each of the first fiber, the second fiber, the third fiber, the fourth fiber, or the fifth fiber) is independently present in an amount of about 1%, about 5%, about 10%, about 15%, about 20%, about 25%, about 30%, about 35%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 65%, about 70%, about 75%, or about 80% w/w of the composition.

In some embodiments, the compositions as described herein include two fibers present in a ratio of about 1:4 to about 4:1, wherein the ratio is the ratio of the first fiber to the second fiber in the composition. For example, a ratio of about 1:3, about 2:5, about 1:2, about 2:3, about 1:1, about 2:1, about 5:2, about 3:1, or about 3:2 of a first fiber to a second fiber. In some embodiments, the compositions include an additional fiber (e.g., a third fiber, a fourth fiber, etc.) present at a ratio of about 1:4 to about 4:1 relative to another fiber in the composition (e.g., the ratio of a first fiber to a third fiber, a second fiber to a third fiber, a third fiber to a fourth fiber, etc. can be any of the ratios of fibers as described herein). For example, a ratio of about 1:3, about 2:5, about 1:2, about 2:3, about 1:1, about 2:1, about 5:2, about 3:1, or about 3:2 of one fiber to another fiber in the composition.

In some embodiments, the compositions as described herein include a first fiber and a second fiber at a ratio of about 1:4 to about 4:1 first fiber:second fiber. For example, about 1:3, about 2:5, about 1:2, about 2:3, about 1:1, about 2:1, about 5:2, about 3:1, or about 3:2 first fiber:second fiber.

In some embodiments, the compositions as described herein include about 2 grams to about 15 grams of the two or more fibers (e.g., the first fiber, the second fiber, the third fiber, the fourth fiber, or the fifth fiber) together. For example, about 2 grams to about 4 grams, about 2 grams to about 6 grams, about 2 grams to about 8 grams, about 2 grams to about 10 grams, about 2 grams to about 12 grams, about 2 grams to about 14 grams, about 4 grams to about 6 grams, about 4 grams to about 8 grams, about 4 grams to about 10 grams, about 4 grams to about 12 grams, about 4 grams to about 14 grams, about 4 grams to about 15 grams, about 6 grams to about 8 grams, about 6 grams to about 10 grams, about 6 grams to about 12 grams, about 6 grams to about 14 grams, about 6 grams to about 15 grams, about 8 grams to about 10 grams, about 8 grams to about 12 grams, about 8 grams to about 14 grams, about 8 grams to about 15 grams, about 10 grams to about 12 grams, about 10 grams to about 14 grams, about 10 grams to about 15 grams, about 11 grams to about 15 grams, about 12 grams to about 14 grams, about 12 grams to about 15 grams, about 13 grams to about 15 grams, or about 14 grams to about 15 grams of the two or more fibers together. In some embodiments, the composition includes about 1 gram to about 3 grams, about 1 gram to about 5 grams, about 1 gram to about 10 grams, about 2 grams to about 4 grams, about 2 grams to about 6 grams, about 3 grams to about 5 grams, about 3 grams to about 6 grams, about 4 grams to about 8 grams, about 4 grams to about 10 grams, about 6 grams to about 10 grams, about 6 grams to about 12 grams, about 8 grams to about 12 grams, or about 8 grams to about 14 grams of the two or more fibers. In some embodiments, the composition includes about 2 grams, about 2.4 grams, about 2.5 grams, about 3 grams, about 3.5 grams, about 3.6 grams, about 4 grams, about 4.2 grams, about 4.5 grams, about 4.8 grams, about 5 grams, about 5.5 grams, about 6 grams, about 6.5 grams, about 7 grams, about 7.5 grams, about 8 grams, about 8.5 grams, about 9 grams, about 9.5 grams, about 10 grams, about 10.5 grams, about 11 grams, about 11.5 grams, about 12 grams, about 12.5 grams, about 13 grams, about 13.5 grams, about 14 grams, about 14.5 grams, or about 15 grams of the two or more fibers together.

In some embodiments, the compositions as described herein include about 0.5 grams to about 10 grams of each of the two or more fibers. For example, about 0.5 grams to about 1 gram, about 0.5 grams to about 2 grams, about 0.5 grams to about 3 grams, about 0.5 grams to about 4 grams, about 0.5 grams to about 5 grams, about 0.5 grams to about 6 grams, about 0.5 grams to about 7 grams, about 0.5 grams to about 8 grams, about 0.5 grams to about 9 grams, about 1 grams to about 2 grams, about 1 grams to about 3 grams, about 1 grams to about 4 grams, about 1 grams to about 5 grams, about 1 grams to about 6 grams, about 1 grams to about 7 grams, about 1 grams to about 8 grams, about 1 grams to about 9 grams, about 1 grams to about 10 grams, about 2 grams to about 3 grams, about 2 grams to about 4 grams, about 2 grams to about 5 grams, about 2 grams to about 6 grams, about 2 grams to about 7 grams, about 2 grams to about 8 grams, about 2 grams to about 9 grams, about 2 grams to about 10 grams, about 3 grams to about 4 grams, about 3 grams to about 5 grams, about 3 grams to about 6 grams, about 3 grams to about 7 grams, about 3 grams to about 8 grams, about 3 grams to about 9 grams, about 3 grams to about 10 grams, about 4 grams to about 5 grams, about 4 grams to about 6 grams, about 4 grams to about 7 grams, about 4 grams to about 8 grams, about 4 grams to about 9 grams, about 4 grams to about 10 grams, about 5 grams to about 6 grams, about 5 grams to about 7 grams, about 5 grams to about 8 grams, about 5 grams to about 9 grams, about 5 grams to about 10 grams, about 6 grams to about 7 grams, about 6 grams to about 8 grams, about 6 grams to about 9 grams, about 6 grams to about 10 grams, about 7 grams to about 8 grams, about 7 grams to about 9 grams, about 7 grams to about 10 grams, about 8 grams to about 9 grams, about 8 grams to about 10 grams, about 9 grams to about 10 grams, about 9 grams to about 10 grams, about 8 grams to about 10 grams, about 7 grams to about 10 grams, about 6 grams to about 10 grams, about 5 grams to about 10 grams, about 4 grams to about 10 grams, about 3 grams to about 10 grams, about 2 grams to about 10 grams, or about 1 gram to about 10 grams of each of the two or more fibers. In some embodiments, the composition includes about 1 gram, 1.5 grams, about 1.8 grams, about 2 grams, about 2.4 grams, about 2.5 grams, about 3 grams, about 3.5 grams, about 4 grams, about 4.5 grams, about 5 grams, about 5.5 grams, about 6 grams, about 6.5 grams, about 7 grams, about 7.5 grams, about 8 grams, about 8.5 grams, about 9 grams, about 9.5 grams, or about 10 grams of each of the two or more fibers.

In some embodiments, a fiber in a composition described herein is present in the same amount (e.g., % w/w of the composition or grams) as at least one other fiber in the composition. In some embodiments, each fiber is present in the same amount (e.g., % w/w of the composition or grams) as at least one other fiber in the composition. In some embodiments, each fiber is present in the same amount (e.g., % w/w of the composition or grams) as the other fiber(s) in the composition. In some embodiments, each fiber is present in a different amount (e.g., % w/w of the composition or grams) from each other fiber(s) in the composition.

In some embodiments, the first fiber, the second fiber, the third fiber, the fourth fiber, or the fifth fiber is each individually selected from the group consisting of: psyllium, pectin, guar, a lignin, cellulose, hemicellulose, resistant starch, a resistant dextrin, inulin, a chitin, pectin, a beta-glucan, potato starch, cornstarch and an oligosaccharide. When both a first and a second fiber are present in a composition provided herein, the first and second fibers are different. When a first, a second, and a third fiber are present in a composition provided herein, the first, second, and third fibers are different from each other. When a first, a second, a third, and a fourth fiber are present in a composition provided herein, the first, second, third, and fourth fibers are different from each other. When a first, a second, a third, a fourth, and a fifth fiber are present in a composition provided herein, the first, second, third, fourth, and fifth fibers are different from each other.

In some embodiments, the fiber (e.g., a first fiber, a second fiber, a third fiber, etc.) in a composition as described herein is a partially hydrolyzed soluble fiber. In some embodiments, the partially hydrolyzed soluble fiber is selected from the group consisting of: partially hydrolyzed psyllium, partially hydrolyzed pectin, partially hydrolyzed wheat dextrin, and a partially hydrolyzed beta-glucan. In some embodiments, the partially hydrolyzed fiber is selected from the group consisting of: partially hydrolyzed psyllium, partially hydrolyzed pectin, partially hydrolyzed guar, a partially hydrolyzed lignin, partially hydrolyzed cellulose, partially hydrolyzed hemicellulose, partially hydrolyzed resistant starch, a partially hydrolyzed resistant dextrin, partially hydrolyzed inulin, a partially hydrolyzed chitin, a partially hydrolyzed pectin, a partially hydrolyzed beta-glucan, a partially hydrolyzed oligosaccharide, and a partially hydrolyzed potato starch.

In some embodiments, the fiber (e.g., a first fiber, a second fiber, a third fiber, etc.) in a composition as described herein comprises less than about 100 ppm gluten. For example, less than about 90, about 80, about 70, about 60, about 50, about 40, about 30, about 20, about 10, or about 5 ppm of gluten. In some embodiments, a composition as described herein comprises about 0 to about 100 ppm of gluten. For example, about 0 to about 5, about 0 to about 10, about 0 to about 15, about 0 to about 20, about 0 to about 25, about 0 to about 30, about 0 to about 35, about 0 to about 40, about 0 to about 45, about 0 to about 50, about 0 to about 55, about 0 to about 60, about 0 to about 65, about 0 to about 70, about 0 to about 75, about 0 to about 80, about 0 to about 85, about 0 to about 90, about 0 to about 95, about 5 to about 10, about 5 to about 15, about 5 to about 20, about 5 to about 25, about 5 to about 30, about 5 to about 35, about 5 to about 40, about 5 to about 45, about 5 to about 50, about 5 to about 55, about 5 to about 60, about 5 to about 65, about 5 to about 70, about 5 to about 75, about 5 to about 80, about 5 to about 85, about 5 to about 90, about 5 to about 95, about 5 to about 100, about 10 to about 15, about 10 to about 20, about 10 to about 25, about 10 to about 30, about 10 to about 35, about 10 to about 40, about 10 to about 45, about 10 to about 50, about 10 to about 55, about 10 to about 60, about 10 to about 65, about 10 to about 70, about 10 to about 75, about 10 to about 80, about 10 to about 85, about 10 to about 90, about 10 to about 95, about 10 to about 100, about 15 to about 20, about 15 to about 25, about 15 to about 30, about 15 to about 35, about 15 to about 40, about 15 to about 45, about 15 to about 50, about 15 to about 55, about 15 to about 60, about 15 to about 65, about 15 to about 70, about 15 to about 75, about 15 to about 80, about 15 to about 85, about 15 to about 90, about 15 to about 95, about 15 to about 100, about 20 to about 25, about 20 to about 30, about 20 to about 35, about 20 to about 40, about 20 to about 45, about 20 to about 50, about 20 to about 55, about 20 to about 60, about 20 to about 65, about 20 to about 70, about 20 to about 75, about 20 to about 80, about 20 to about 85, about 20 to about 90, about 20 to about 95, about 20 to about 100, about 25 to about 30, about 25 to about 35, about 25 to about 40, about 25 to about 45, about 25 to about 50, about 25 to about 55, about 25 to about 60, about 25 to about 65, about 25 to about 70, about 25 to about 75, about 25 to about 80, about 25 to about 85, about 25 to about 90, about 25 to about 95, about 25 to about 100, about 30 to about 35, about 30 to about 40, about 30 to about 45, about 30 to about 50, about 30 to about 55, about 30 to about 60, about 30 to about 65, about 30 to about 70, about 30 to about 75, about 30 to about 80, about 30 to about 85, about 30 to about 90, about 30 to about 95, about 30 to about 100, about 35 to about 40, about 35 to about 45, about 35 to about 50, about 35 to about 55, about 35 to about 60, about 35 to about 65, about 35 to about 70, about 35 to about 75, about 35 to about 80, about 35 to about 85, about 35 to about 90, about 35 to about 95, about 35 to about 100, about 40 to about 45, about 40 to about 50, about 40 to about 55, about 40 to about 60, about 40 to about 65, about 40 to about 70, about 40 to about 75, about 40 to about 80, about 40 to about 85, about 40 to about 90, about 40 to about 95, about 40 to about 100, about 45 to about 50, about 45 to about 55, about 45 to about 60, about 45 to about 65, about 45 to about 70, about 45 to about 75, about 45 to about 80, about 45 to about 85, about 45 to about 90, about 45 to about 95, about 45 to about 100, about 50 to about 55, about 50 to about 60, about 50 to about 65, about 50 to about 70, about 50 to about 75, about 50 to about 80, about 50 to about 85, about 50 to about 90, about 50 to about 95, about 50 to about 100, about 55 to about 60, about 55 to about 65, about 55 to about 70, about 55 to about 75, about 55 to about 80, about 55 to about 85, about 55 to about 90, about 55 to about 95, about 55 to about 100, about 60 to about 65, about 60 to about 70, about 60 to about 75, about 60 to about 80, about 60 to about 85, about 60 to about 90, about 60 to about 95, about 60 to about 100, about 65 to about 70, about 65 to about 75, about 65 to about 80, about 65 to about 85, about 65 to about 90, about 65 to about 95, about 65 to about 100, about 70 to about 75, about 70 to about 80, about 70 to about 85, about 70 to about 90, about 70 to about 95, about 70 to about 100, about 75 to about 80, about 75 to about 85, about 75 to about 90, about 75 to about 95, about 75 to about 100, about 80 to about 85, about 80 to about 90, about 80 to about 95, about 80 to about 100, about 85 to about 90, about 85 to about 95, about 85 to about 100, about 90 to about 95, about 90 to about 100, or about 95 to about 100 ppm of gluten. In some embodiments, the fiber is wheat dextrin. In some embodiments, the fiber is partially hydrolyzed wheat dextrin.

In some embodiments, the fiber (e.g., a first fiber, a second fiber, a third fiber, etc.) in a composition as described herein is “gluten-free.” As used herein, a “gluten-free” fiber or composition is a fiber or composition that comprises less than about 20 ppm of gluten. For example, a fiber or composition that comprises about 0 to about 5, about 0 to about 10, about 0 to about 15, about 0 to about 19, about 5 to about 10, about 5 to about 15, about 5 to about 19, about 10 to about 15, about 10 to about 19, or about 15 to about 19 ppm of gluten. In some embodiments, the wheat dextrin is gluten free. In some embodiments, the partially hydrolyzed wheat dextrin is gluten free.

The quantity of gluten in a fiber or composition can be determined using methods known in the art. Such methods can include those described in Miranda-Castro et al., Harnessing Aptamers to Overcome Challenges in Gluten Detection, Biosensors (Basel). 2016; 6(2):16.

In some embodiments, wherein the composition includes two fibers, the first fiber is wheat dextrin and the second fiber is selected from psyllium, inulin, potato starch or cornstarch. In some embodiments, wherein the composition includes two fibers, the first fiber is psyllium and the second fiber is selected from wheat dextrin, inulin, potato starch or cornstarch. In some embodiments, wherein the composition includes two fibers, the first fiber is inulin and the second fiber is selected from wheat dextrin, psyllium, potato starch or cornstarch. In some embodiments, wherein the composition includes two fibers, the first fiber is potato starch and the second fiber is selected from wheat dextrin, psyllium, inulin or cornstarch. In some embodiments, wherein the composition includes two fibers, the first fiber is beta-glucan and the second fiber is selected from wheat dextrin, psyllium, inulin, potato starch or cornstarch.

In some embodiments, a composition as described herein comprises a first fiber and a second fiber. In some embodiments, the first fiber is present in an amount of about 20% to about 80% w/w of the composition and the second fiber is present in an amount of about 20% to about 80% w/w of the composition.

In some embodiments, wherein the composition includes two fibers, the first fiber is an oligosaccharide and the second fiber is selected from the group consisting of: pectin, inulin, psyllium, guar, a lignin, cellulose, hemicellulose, resistant starch, a resistant dextrin, a chitin, a beta-glucan, potato starch, and a combination thereof. In some embodiments, the second fiber is pectin, inulin, wheat dextrin, or potato starch. In some embodiments, the oligosaccharide is selected from the group consisting of: fructooligosaccharide, maltose, iso-maltose, a maltosyl-iso-malto-oligosaccharide, and a combination thereof. In some embodiments, the oligosaccharide is fructooligosaccharide. In some embodiments, the oligosaccharide is maltose.

In some embodiments, wherein the composition includes two fibers, the first fiber is psyllium and the second fiber is wheat dextrin. In some embodiments, wherein the composition includes two fibers, the first fiber is psyllium and the second fiber is inulin. In some embodiments, wherein the composition includes two fibers, the first fiber is wheat dextrin and the second fiber is inulin.

In some embodiments, the composition includes psyllium present in an amount of about 45% to about 55% w/w of the composition and wheat dextrin present in an amount of about 45% to about 55% w/w of the composition.

In some embodiments, the composition includes psyllium present in an amount of about 45% to about 55% w/w of the composition and inulin present in an amount of about 45% to about 55% w/w of the composition.

In some embodiments, the composition includes wheat dextrin present in an amount of about 45% to about 55% w/w of the composition and inulin present in an amount of about 45% to about 55% w/w of the composition.

In some embodiments, a composition as described herein includes psyllium present in an amount of about 50% w/w of the composition and wheat dextrin present in an amount of about 50% w/w of the composition.

In some embodiments, a composition as described herein includes psyllium present in an amount of about 50% w/w of the composition and inulin present in an amount of about 50% w/w of the composition.

In some embodiments, a composition as described herein includes wheat dextrin present in an amount of about 50% w/w of the composition and inulin present in an amount of about 50% w/w of the composition.

In some embodiments, wherein the composition includes two fibers, the first fiber is beta-glucan and the second fiber is wheat dextrin. In some embodiments, wherein the composition includes two fibers, the first fiber is beta-glucan and the second fiber is psyllium.

In some embodiments, the composition includes beta-glucan present in an amount of about 45% to about 55% w/w of the composition and wheat dextrin present in an amount of about 45% to about 55% w/w of the composition.

In some embodiments, the composition includes beta-glucan present in an amount of about 45% to about 55% w/w of the composition and psyllium present in an amount of about 45% to about 55% w/w of the composition.

In some embodiments, a composition as described herein includes beta-glucan present in an amount of about 50% w/w of the composition and wheat dextrin present in an amount of about 50% w/w of the composition.

In some embodiments, a composition as described herein includes beta-glucan present in an amount of about 50% w/w of the composition and psyllium present in an amount of about 50% w/w of the composition.

In some embodiments, wherein the composition includes two fibers, the composition further includes a third fiber. In some embodiments, the third fiber is present in an amount of about 20% to about 40% w/w of the composition. In some embodiments, the third fiber is present in an amount of about 1% to about 10% w/w of the composition.

In some embodiments, the ratio of the first fiber to third fiber is about 1:2 to about 10:1 first fiber:third fiber. For example, about 2:3, about 1:1, about 2:1, about 5:2, about 3:1, or about 3:2 first fiber:second fiber. In some embodiments, the ratio of the first fiber to third fiber is about 5:1 or about 10:1 first fiber:third fiber. In some embodiments, the ratio of the first fiber to second fiber to third fiber is about 1:1:1 first fiber:second fiber:third fiber.

In some embodiments, the third fiber is an oligosaccharide is selected from the group consisting of: fructooligosaccharide, maltose, iso-maltose, a maltosyl-iso-malto-oligosaccharide, and a combination thereof. In some embodiments, the oligosaccharide is fructooligosaccharide. In some embodiments, the oligosaccharide is maltose.

In some embodiments, wherein the composition includes three fibers, the first fiber is beta-glucan, the second fiber is wheat dextrin, and the third fiber is psyllium.

In some embodiments, a composition as described herein comprises less than about 100 ppm gluten. For example, less than about 90, about 80, about 70, about 60, about 50, about 40, about 30, about 20, about 10, or about 5 ppm of gluten. In some embodiments, a composition as described herein comprises about 0 to about 100 ppm of gluten. For example, about 0 to about 5, about 0 to about 10, about 0 to about 15, about 0 to about 20, about 0 to about 25, about 0 to about 30, about 0 to about 35, about 0 to about 40, about 0 to about 45, about 0 to about 50, about 0 to about 55, about 0 to about 60, about 0 to about 65, about 0 to about 70, about 0 to about 75, about 0 to about 80, about 0 to about 85, about 0 to about 90, about 0 to about 95, about 5 to about 10, about 5 to about 15, about 5 to about 20, about 5 to about 25, about 5 to about 30, about 5 to about 35, about 5 to about 40, about 5 to about 45, about 5 to about 50, about 5 to about 55, about 5 to about 60, about 5 to about 65, about 5 to about 70, about 5 to about 75, about 5 to about 80, about 5 to about 85, about 5 to about 90, about 5 to about 95, about 5 to about 100, about 10 to about 15, about 10 to about 20, about 10 to about 25, about 10 to about 30, about 10 to about 35, about 10 to about 40, about 10 to about 45, about 10 to about 50, about 10 to about 55, about 10 to about 60, about 10 to about 65, about 10 to about 70, about 10 to about 75, about 10 to about 80, about 10 to about 85, about 10 to about 90, about 10 to about 95, about 10 to about 100, about 15 to about 20, about 15 to about 25, about 15 to about 30, about 15 to about 35, about 15 to about 40, about 15 to about 45, about 15 to about 50, about 15 to about 55, about 15 to about 60, about 15 to about 65, about 15 to about 70, about 15 to about 75, about 15 to about 80, about 15 to about 85, about 15 to about 90, about 15 to about 95, about 15 to about 100, about 20 to about 25, about 20 to about 30, about 20 to about 35, about 20 to about 40, about 20 to about 45, about 20 to about 50, about 20 to about 55, about 20 to about 60, about 20 to about 65, about 20 to about 70, about 20 to about 75, about 20 to about 80, about 20 to about 85, about 20 to about 90, about 20 to about 95, about 20 to about 100, about 25 to about 30, about 25 to about 35, about 25 to about 40, about 25 to about 45, about 25 to about 50, about 25 to about 55, about 25 to about 60, about 25 to about 65, about 25 to about 70, about 25 to about 75, about 25 to about 80, about 25 to about 85, about 25 to about 90, about 25 to about 95, about 25 to about 100, about 30 to about 35, about 30 to about 40, about 30 to about 45, about 30 to about 50, about 30 to about 55, about 30 to about 60, about 30 to about 65, about 30 to about 70, about 30 to about 75, about 30 to about 80, about 30 to about 85, about 30 to about 90, about 30 to about 95, about 30 to about 100, about 35 to about 40, about 35 to about 45, about 35 to about 50, about 35 to about 55, about 35 to about 60, about 35 to about 65, about 35 to about 70, about 35 to about 75, about 35 to about 80, about 35 to about 85, about 35 to about 90, about 35 to about 95, about 35 to about 100, about 40 to about 45, about 40 to about 50, about 40 to about 55, about 40 to about 60, about 40 to about 65, about 40 to about 70, about 40 to about 75, about 40 to about 80, about 40 to about 85, about 40 to about 90, about 40 to about 95, about 40 to about 100, about 45 to about 50, about 45 to about 55, about 45 to about 60, about 45 to about 65, about 45 to about 70, about 45 to about 75, about 45 to about 80, about 45 to about 85, about 45 to about 90, about 45 to about 95, about 45 to about 100, about 50 to about 55, about 50 to about 60, about 50 to about 65, about 50 to about 70, about 50 to about 75, about 50 to about 80, about 50 to about 85, about 50 to about 90, about 50 to about 95, about 50 to about 100, about 55 to about 60, about 55 to about 65, about 55 to about 70, about 55 to about 75, about 55 to about 80, about 55 to about 85, about 55 to about 90, about 55 to about 95, about 55 to about 100, about 60 to about 65, about 60 to about 70, about 60 to about 75, about 60 to about 80, about 60 to about 85, about 60 to about 90, about 60 to about 95, about 60 to about 100, about 65 to about 70, about 65 to about 75, about 65 to about 80, about 65 to about 85, about 65 to about 90, about 65 to about 95, about 65 to about 100, about 70 to about 75, about 70 to about 80, about 70 to about 85, about 70 to about 90, about 70 to about 95, about 70 to about 100, about 75 to about 80, about 75 to about 85, about 75 to about 90, about 75 to about 95, about 75 to about 100, about 80 to about 85, about 80 to about 90, about 80 to about 95, about 80 to about 100, about 85 to about 90, about 85 to about 95, about 85 to about 100, about 90 to about 95, about 90 to about 100, or about 95 to about 100 ppm of gluten.

In some embodiments, a composition as described herein is gluten-free.

In some embodiments, a composition as described herein can further include a preservative. A “preservative” as used herein refers to an agent that protects against decay, discoloration, and/or spoilage. Non-limiting examples of a preservative include ascorbic acid, an ascorbate, a palmitate, citric acid, a benzoate, benzoic acid, a propionate, propionic acid, a sorbate, sorbic acid, an erythorbate, a nitrite, ethylenediaminetetraacetic acid (EDTA), butylated hydroxyanisole (BHA), butylated hydroxytoluene (BHT), capryllic acid, dilauryl thiodipropionate, erythorbic acid, gum guaiac, methylparaben, a sulfite, a bisulfate, a metabisulfite, propyl gallatepy, propylparaben, stannous chloride, sulfur dioxide, thiodipropionic acid, and a tocopherol.

In some embodiments, the preservative is present in an amount of about 0.01% to about 2% w/w of the composition. In some embodiments, the preservative is present in an amount of about 0.01% to about 1.5%, about 0.01% to about 1.0%, about 0.01% to about 0.5%, about 0.01% to about 0.1%, about 0.01% to about 0.05%, about 1.5% to about 2%, about 1% to about 2%, about 0.5% to about 2%, about 0.1% to about 2%, about 0.05% to about 2%, about 0.01% to about 0.8%, about 0.8% to about 2%, or about 0.1% to about 0.9% w/w of the composition. For example, about 0.05% to about 0.4%, about 0.05% to about 0.2%, about 0.2% to about 0.5%, about 0.15% to about 0.25%, or about 0.1% to about 0.3% w/w of the composition. In some embodiments, the preservative is present in an amount of about 0.01%, about 0.05%, about 0.1%, about 0.12%, about 0.14%, about 0.15%, about 0.16%, about 0.18%, about 0.2%, about 0.22% about 0.25%, about 0.26%, about 0.28%, about 0.3%, about 0.32%, about 0.34%, about 0.35%, about 0.36%, about 0.38%, about 0.4%, about 0.45%, about 0.5%, about 0.6%, about 0.65%, about 0.7%, about 0.8%, about 0.9%, about 1%, about 1.5%, or about 2% w/w of the composition.

In some embodiments, a composition as described herein can further include a sweetener. Non-limiting examples of a sweetener include sorbitol, sucrose, a saccharin, a cyclamate, aspartame, sucralose, thaumatin, acesulfam K, xylose, ribose, glucose, mannose, galactose, fructose, dextrose, sucrose, maltose, starch or corn syrup, sorbitol, xylitol, mannitol, a steviol glycoside and glycerin. In some embodiments, a steviol glycoside includes stevioside and rebaudioside (e.g., STEVIA® sweetener). A sweetener can be an intense sweetener or a natural sugar.

In some embodiments, the sweetener is present in an amount of about 0.001% to about 20% w/w of the composition. For example, about 0.001% to about 0.05%, about 0.01% to about 0.5%, about 0.1% to about 1.5%, about 1% to about 5%, about 5% to about 10%, about 10% to about 15%, about 15% to about 20% w/w of the composition.

In some embodiments, a composition as described herein can further include a flavoring agent. For example, the composition can further include a flavor enhancer. Non-limiting examples of flavoring agents include a natural flavoring agent (e.g., a substance obtained from a plant or animal raw material by physical, microbiological, or enzymatic processes), a natural fruit flavoring agent (e.g., a substance obtained from a fruit raw material by physical, microbiological, or enzymatic processes), an artificial flavoring agent, an artificial fruit-flavoring agent, and a flavor enhancer. In some embodiments, the flavoring agent is selected from the group consisting of: almond oil, DL-menthol, ethyl acetate, ethyl vanillin, L-menthol, methyl salicylate, peppermint oil, peppermint spirit, thymol, vanillin, cinnamomum, sodium glutamate, eucalyptus, acacia, anise oil, caraway oil, cardamom, cherry syrup, citric acid syrup, clove oil, cocoa, coriander oil, fennel oil, ginger, glycerin, glycerrhiza, honey, lavender oil, lemon oil, mannitol, nutmeg oil, orange oil, orange flower water, raspberry, rose oil, rosemary oil, sarsaparilla syrup, spearmint oil, thyme oil, tolu balsam syrup, wild cherry syrup, and a combination thereof. In some embodiments, the flavoring agent is selected from the group consisting of: almond oil, ethyl vanillin, L-menthol, methyl salicylate, peppermint oil, vanillin, cinnamon, cherry syrup, cocoa, glycerin, honey, lemon oil, and a combination thereof.

In some embodiments, the flavoring agent is present in an amount of about 0.01% to about 2% w/w of the composition.

In some embodiments, the composition further includes an acid. Non-limiting examples of an acid include HCl, HI, HBr, HClO₄, HClO₃, HNO₃, H₂SO₄, phosphoric acid, phosphorous acid, acetic acid, oxalic acid, ascorbic acid, carbonic acid, sulfurous acid, tartaric acid, citric acid, malonic acid, phthalic acid, barbituric acid, cinnamic acid, glutaric acid, hexanoic acid, malic acid, folic acid, propionic acid, stearic acid, trifluoroacetic acid, acetylsalicylic acid, glutamic acid, azelaic acid, benzilic acid, fumaric acid, gluconic acid, lactic acid, oleic acid, propiolic acid, rosolic acid, tannic acid, uric acid, gallic acid, and a combination thereof.

In some embodiments, the acid is a strong acid. In some embodiments, the strong acid is selected from the group consisting of: HCl, HI, HBr, HClO₄, HClO₃, HNO₃, H₂SO₄, and a combination thereof. In some embodiments, the acid is a weak acid. In some embodiments, the weak acid is selected from the group consisting of: phosphorous acid, acetic acid, oxalic acid, phosphoric acid, ascorbic acid, carbonic acid, citric acid, sulfurous acid, tartaric acid, malonic acid, phthalic acid, barbituric acid, cinnamic acid, glutaric acid, hexanoic acid, malic acid, folic acid, propionic acid, stearic acid, trifluoroacetic acid, acetylsalicylic acid, glutamic acid, azelaic acid, benzilic acid, fumaric acid, gluconic acid, lactic acid, oleic acid, propiolic acid, rosolic acid, tannic acid, uric acid, gallic acid, and a combination thereof.

The compositions described herein can be formulated for oral delivery in a variety of ways. For example, the composition can be in the form of a tablet, powder or gummy (e.g., a soft, chewable gummy). In some embodiments, a composition as provided herein is a solid composition. In some embodiments, a composition as provided herein is a powder. For oral administration, tablets or capsules can be prepared by conventional means with pharmaceutically acceptable excipients such as binding agents, fillers, lubricants, disintegrants, or wetting agents. The tablets can be coated by methods known in the art. Liquid preparations for oral administration can take the form of, for example, solutions, syrups, or suspension, or they can be presented as a dry product for constitution with saline or other suitable liquid vehicle before use. For example, compositions as described herein can be presented as dry powder and dissolved in a suitable liquid carrier. In some embodiments, a capsule can be a delayed-release capsule.

In some embodiments, the compositions described herein can be packaged in the form of one or more dosage forms (e.g., a packet, a gummy, a tablet, and a capsule) to have a unit dosage equal to the desired dosage for a particular subject. For example, each dosage form can include about 2 grams to about 15 grams by weight of a composition described herein. In some embodiments, a total daily dose may be prepared and administered in the form of one or more dosage forms (e.g., two packets, three packets, four packets, five packets, or six packets).

In some embodiments, each dosage form can include about 2 grams to about 4 grams, about 2 grams to about 6 grams, about 2 grams to about 8 grams, about 2 grams to about 10 grams, about 2 grams to about 12 grams, about 2 grams to about 14 grams, about 4 grams to about 6 grams, about 4 grams to about 8 grams, about 4 grams to about 10 grams, about 4 grams to about 12 grams, about 4 grams to about 14 grams, about 4 grams to about 15 grams, about 6 grams to about 8 grams, about 6 grams to about 10 grams, about 6 grams to about 12 grams, about 6 grams to about 14 grams, about 6 grams to about 15 grams, about 8 grams to about 10 grams, about 8 grams to about 12 grams, about 8 grams to about 14 grams, about 8 grams to about 15 grams, about 10 grams to about 12 grams, about 10 grams to about 14 grams, about 10 grams to about 15 grams, about 11 grams to about 15 grams, about 12 grams to about 14 grams, about 12 grams to about 15 grams, about 13 grams to about 15 grams, or about 14 grams to about 15 grams of the compositions described herein. In some embodiments, each dosage form can include about 1 gram to about 3 grams, about 1 gram to about 5 grams, about 1 gram to about 10 grams, about 2 grams to about 4 grams, about 2 grams to about 6 grams, about 3 grams to about 5 grams, about 3 grams to about 6 grams, about 4 grams to about 8 grams, about 4 grams to about 10 grams, about 6 grams to about 10 grams, about 6 grams to about 12 grams, about 8 grams to about 12 grams, or about 8 grams to about 14 grams of the compositions described herein. In some embodiments, each dosage form can include about 2 grams, about 2.4 grams, about 2.5 grams, about 3 grams, about 3.5 grams, about 3.6 grams, about 4 grams, about 4.2 grams, about 4.5 grams, about 4.8 grams, about 5 grams, about 5.5 grams, about 6 grams, about 6.5 grams, about 7 grams, about 7.5 grams, about 8 grams, about 8.5 grams, about 9 grams, about 9.5 grams, about 10 grams, about 10.5 grams, about 11 grams, about 11.5 grams, about 12 grams, about 12.5 grams, about 13 grams, about 13.5 grams, about 14 grams, about 14.5 grams, or about 15 grams of the compositions described herein.

In some embodiments, a dosage form can include a first fiber present in an amount of about 1 to about 4 grams and a second fiber present in an amount of about 1 to about 4 grams. In some embodiments, a dosage form can include a first fiber present in an amount of about 2 to about 3 grams and a second fiber present in an amount of about 2 to about 3 grams. In some embodiments, a dosage form can include a first fiber present in an amount of about 2 to about 3 grams and a second fiber present in an amount of about 2 grams. In some embodiments, a dosage form can include a first fiber present in an amount of about 2 grams and a second fiber present in an amount of about 2 grams. In some embodiments, the first fiber is psyllium and the second fiber is wheat dextrin. In some embodiments, first fiber is psyllium and the second fiber is inulin. In some embodiments, the first fiber is wheat dextrin and the second fiber is inulin. In some embodiments, the first fiber is beta-glucan and the second fiber is wheat dextrin. In some embodiments, the first fiber is beta-glucan and the second fiber is psyllium.

The unit dosages of a particular composition will depend on many factors including the mode of administration or the needs of the subject taking the composition. As an example, a composition as described herein can be formulated in a dose such that an individual receives the amounts as shown in Table 1, e.g., as a powder or as a powder dissolved in a beverage.

TABLE 1

| | Dosage Amount |
|---|---|
| Components | (grams) |
| First fiber (e.g., psyllium) | 2 |
| Second fiber (e.g., wheat dextrin | 2 |
| or inulin) | |

In some embodiments, a composition as provided herein is added to a food. For example, the composition can be added to cereal, yogurt, pudding, and gummies. In some embodiments, the composition is added to a beverage. For example, the compositions described herein can be added to milk, juice, or lemonade. In some embodiments, a composition as provided herein is added to a food containing probiotics. Non-limiting examples of foods that can contain probiotics include fermented foods such as kefir, kombucha, yogurt, sauerkraut, and kimchi. In some embodiments, a composition described herein is added to kefir or kombucha.

### Kits

Also provided herein are kits for a dietary fiber combination, e.g., a kit, comprising i) a first fiber; and ii) a second fiber. In some embodiments, the kit includes i) a first fiber; ii) a second fiber; and iii) a third fiber. In some embodiments, the kit includes i) a first fiber; ii) a second fiber; iii) a third fiber, and iv) a fourth fiber. In some embodiments, the fibers (e.g., a first fiber and a second fiber) are present as a composition. In some embodiments, each fiber is present as an independent composition from each other fiber.

In some embodiments, the kit includes about 20% to about 80% w/w of the dietary fiber combination of each of the fibers (e.g., each of the first fiber, the second fiber, the third fiber, the fourth fiber, or the fifth fiber) independently. For example, about 20% to about 70%, about 20% to about 60%, about 20% to about 50%, about 20% to about 40%, about 20% to about 30%, about 30% to about 80%, about 30% to about 70%, about 30% to about 60%, about 30% to about 50%, about 30% to about 40%, about 40% to about 80%, about 40% to about 70%, about 40% to about 60%, about 40% to about 50%, about 50% to about 80%, about 50% to about 70%, about 50% to about 60%, about 60% to about 80%, about 60% to about 70%, or about 70% to about 80% w/w of the dietary fiber combination of each of the fibers (e.g., each of the first fiber, the second fiber, the third fiber, the fourth fiber, or the fifth fiber) independently. In some embodiments, the kit includes about 35% to about 45%, about 45% to about 55%, about 35% to about 40%, about 40% to about 45%, about 45% to about 50%, about 50% to about 55%, about 55% to about 60%, or about 60% to about 65% w/w of the dietary fiber combination of each of the fibers (e.g., each of the first fiber, the second fiber, the third fiber, the fourth fiber, or the fifth fiber) independently. In some embodiments, the kit includes about 20%, about 25%, about 30%, about 35%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 65%, about 70%, about 75%, or about 80% w/w of the dietary fiber combination of each of the fibers (e.g., each of the first fiber, the second fiber, the third fiber, the fourth fiber, or the fifth fiber) independently.

In some embodiments, the kit includes about 20% to about 80% w/w of the composition (e.g., a composition comprising a first and second fiber) of each of the fibers (e.g., each of the first fiber, the second fiber, the third fiber, the fourth fiber, or the fifth fiber) independently. For example, about 20% to about 70%, about 20% to about 60%, about 20% to about 50%, about 20% to about 40%, about 20% to about 30%, about 30% to about 80%, about 30% to about 70%, about 30% to about 60%, about 30% to about 50%, about 30% to about 40%, about 40% to about 80%, about 40% to about 70%, about 40% to about 60%, about 40% to about 50%, about 50% to about 80%, about 50% to about 70%, about 50% to about 60%, about 60% to about 80%, about 60% to about 70%, or about 70% to about 80% w/w of the composition of each of the fibers (e.g., each of the first fiber, the second fiber, the third fiber, the fourth fiber, or the fifth fiber) independently. In some embodiments, the kit includes about 35% to about 45%, about 45% to about 55%, about 35% to about 40%, about 40% to about 45%, about 45% to about 50%, about 50% to about 55%, about 55% to about 60%, or about 60% to about 65% w/w of the composition of each of the fibers (e.g., each of the first fiber, the second fiber, the third fiber, the fourth fiber, or the fifth fiber) independently. In some embodiments, the kit includes about 20%, about 25%, about 30%, about 35%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 65%, about 70%, about 75%, or about 80% w/w of the composition of each of the fibers (e.g., each of the first fiber, the second fiber, the third fiber, the fourth fiber, or the fifth fiber) independently.

In some embodiments, the kit includes two fibers present in a ratio of about 1:4 to about 4:1, wherein the ratio is the ratio of the first fiber to the second fiber in the composition. For example, a ratio of about 1:3, about 2:5, about 1:2, about 2:3, about 1:1, about 2:1, about 5:2, about 3:1, or about 3:2 of a first fiber to a second fiber. In some embodiments, the kits as described herein can include an additional fiber (e.g., a third fiber, a fourth fiber, etc.) present at a ratio of about 1:4 to about 4:1 relative to another fiber in the composition (e.g., the ratio of a first fiber to a third fiber, a second fiber to a third fiber, a third fiber to a fourth fiber, etc. can be any of the ratios of fibers as described herein). For example, a ratio of about 1:3, about 2:5, about 1:2, about 2:3, about 1:1, about 2:1, about 5:2, about 3:1, or about 3:2 of one fiber to another fiber in the composition.

In some embodiments, the kits as described herein can include a first fiber and a second fiber at a ratio of about 1:4 to about 4:1 first fiber:second fiber. For example, about 1:3, about 2:5, about 1:2, about 2:3, about 1:1, about 2:1, about 5:2, about 3:1, or about 3:2 first fiber:second fiber.

In some embodiments, the kits as described herein include about 2 grams to about 15 grams of the two or more fibers (e.g., the first fiber, the second fiber, the third fiber, the fourth fiber, or the fifth fiber) together. For example, about 2 grams to about 4 grams, about 2 grams to about 6 grams, about 2 grams to about 8 grams, about 2 grams to about 10 grams, about 2 grams to about 12 grams, about 2 grams to about 14 grams, about 4 grams to about 6 grams, about 4 grams to about 8 grams, about 4 grams to about 10 grams, about 4 grams to about 12 grams, about 4 grams to about 14 grams, about 4 grams to about 15 grams, about 6 grams to about 8 grams, about 6 grams to about 10 grams, about 6 grams to about 12 grams, about 6 grams to about 14 grams, about 6 grams to about 15 grams, about 8 grams to about 10 grams, about 8 grams to about 12 grams, about 8 grams to about 14 grams, about 8 grams to about 15 grams, about 10 grams to about 12 grams, about 10 grams to about 14 grams, about 10 grams to about 15 grams, about 11 grams to about 15 grams, about 12 grams to about 14 grams, about 12 grams to about 15 grams, about 13 grams to about 15 grams, or about 14 grams to about 15 grams of the two or more fibers together. In some embodiments, the kit includes about 1 gram to about 3 grams, about 1 gram to about 5 grams, about 1 gram to about 10 grams, about 2 grams to about 4 grams, about 2 grams to about 6 grams, about 3 grams to about 5 grams, about 3 grams to about 6 grams, about 4 grams to about 8 grams, about 4 grams to about 10 grams, about 6 grams to about 10 grams, about 6 grams to about 12 grams, about 8 grams to about 12 grams, or about 8 grams to about 14 grams of the two or more fibers. In some embodiments, the composition includes about 2 grams, about 2.4 grams, about 2.5 grams, about 3 grams, about 3.5 grams, about 3.6 grams, about 4 grams, about 4.2 grams, about 4.5 grams, about 4.8 grams, about 5 grams, about 5.5 grams, about 6 grams, about 6.5 grams, about 7 grams, about 7.5 grams, about 8 grams, about 8.5 grams, about 9 grams, about 9.5 grams, about 10 grams, about 10.5 grams, about 11 grams, about 11.5 grams, about 12 grams, about 12.5 grams, about 13 grams, about 13.5 grams, about 14 grams, about 14.5 grams, or about 15 grams of the two or more fibers together.

In some embodiments, the kit includes about 0.5 grams to about 10 grams of each of the two or more fibers. For example, about 0.5 grams to about 1 gram, about 0.5 grams to about 2 grams, about 0.5 grams to about 3 grams, about 0.5 grams to about 4 grams, about 0.5 grams to about 5 grams, about 0.5 grams to about 6 grams, about 0.5 grams to about 7 grams, about 0.5 grams to about 8 grams, about 0.5 grams to about 9 grams, about 1 grams to about 2 grams, about 1 grams to about 3 grams, about 1 grams to about 4 grams, about 1 grams to about 5 grams, about 1 grams to about 6 grams, about 1 grams to about 7 grams, about 1 grams to about 8 grams, about 1 grams to about 9 grams, about 1 grams to about 10 grams, about 2 grams to about 3 grams, about 2 grams to about 4 grams, about 2 grams to about 5 grams, about 2 grams to about 6 grams, about 2 grams to about 7 grams, about 2 grams to about 8 grams, about 2 grams to about 9 grams, about 2 grams to about 10 grams, about 3 grams to about 4 grams, about 3 grams to about 5 grams, about 3 grams to about 6 grams, about 3 grams to about 7 grams, about 3 grams to about 8 grams, about 3 grams to about 9 grams, about 3 grams to about 10 grams, about 4 grams to about 5 grams, about 4 grams to about 6 grams, about 4 grams to about 7 grams, about 4 grams to about 8 grams, about 4 grams to about 9 grams, about 4 grams to about 10 grams, about 5 grams to about 6 grams, about 5 grams to about 7 grams, about 5 grams to about 8 grams, about 5 grams to about 9 grams, about 5 grams to about 10 grams, about 6 grams to about 7 grams, about 6 grams to about 8 grams, about 6 grams to about 9 grams, about 6 grams to about 10 grams, about 7 grams to about 8 grams, about 7 grams to about 9 grams, about 7 grams to about 10 grams, about 8 grams to about 9 grams, about 8 grams to about 10 grams, about 9 grams to about 10 grams, about 9 grams to about 10 grams, about 8 grams to about 10 grams, about 7 grams to about 10 grams, about 6 grams to about 10 grams, about 5 grams to about 10 grams, about 4 grams to about 10 grams, about 3 grams to about 10 grams, about 2 grams to about 10 grams, or about 1 gram to about 10 grams of each of the two or more fibers. In some embodiments, the kit includes about 1 gram, 1.5 grams, about 1.8 grams, about 2 grams, about 2.4 grams, about 2.5 grams, about 3 grams, about 3.5 grams, about 4 grams, about 4.5 grams, about 5 grams, about 5.5 grams, about 6 grams, about 6.5 grams, about 7 grams, about 7.5 grams, about 8 grams, about 8.5 grams, about 9 grams, about 9.5 grams, or about 10 grams of each of the two or more fibers.

In some embodiments, the kits as described herein include dosage forms (e.g., packets, gummies, tablets, and capsules) containing about 2 grams to about 15 grams of two or more fibers (e.g., a first fiber, a second fiber, a third fiber, a fourth fiber, or a fifth fiber) together. For example, about 2 grams to about 4 grams, about 2 grams to about 6 grams, about 2 grams to about 8 grams, about 2 grams to about 10 grams, about 2 grams to about 12 grams, about 2 grams to about 14 grams, about 4 grams to about 6 grams, about 4 grams to about 8 grams, about 4 grams to about 10 grams, about 4 grams to about 12 grams, about 4 grams to about 14 grams, about 4 grams to about 15 grams, about 6 grams to about 8 grams, about 6 grams to about 10 grams, about 6 grams to about 12 grams, about 6 grams to about 14 grams, about 6 grams to about 15 grams, about 8 grams to about 10 grams, about 8 grams to about 12 grams, about 8 grams to about 14 grams, about 8 grams to about 15 grams, about 10 grams to about 12 grams, about 10 grams to about 14 grams, about 10 grams to about 15 grams, about 11 grams to about 15 grams, about 12 grams to about 14 grams, about 12 grams to about 15 grams, about 13 grams to about 15 grams, or about 14 grams to about 15 grams of the two or more fibers together. In some embodiments, the kit includes include dosage forms (e.g., packets, gummies, tablets, and capsules) containing about 1 gram to about 3 grams, about 1 gram to about 5 grams, about 1 gram to about 10 grams, about 2 grams to about 4 grams, about 2 grams to about 6 grams, about 3 grams to about 5 grams, about 3 grams to about 6 grams, about 4 grams to about 8 grams, about 4 grams to about 10 grams, about 6 grams to about 10 grams, about 6 grams to about 12 grams, about 8 grams to about 12 grams, or about 8 grams to about 14 grams of the two or more fibers. In some embodiments, the kit includes dosage forms (e.g., packets, gummies, tablets, and capsules) containing about 2 grams, about 2.4 grams, about 2.5 grams, about 3 grams, about 3.5 grams, about 3.6 grams, about 4 grams, about 4.2 grams, about 4.5 grams, about 4.8 grams, about 5 grams, about 5.5 grams, about 6 grams, about 6.5 grams, about 7 grams, about 7.5 grams, about 8 grams, about 8.5 grams, about 9 grams, about 9.5 grams, about 10 grams, about 10.5 grams, about 11 grams, about 11.5 grams, about 12 grams, about 12.5 grams, about 13 grams, about 13.5 grams, about 14 grams, about 14.5 grams, or about 15 grams of the two or more fibers together.

In some embodiments, the kit includes include dosage forms (e.g., packets, gummies, tablets, and capsules) containing about 0.5 grams to about 10 grams of each of the two or more fibers. For example, about 0.5 grams to about 1 gram, about 0.5 grams to about 2 grams, about 0.5 grams to about 3 grams, about 0.5 grams to about 4 grams, about 0.5 grams to about 5 grams, about 0.5 grams to about 6 grams, about 0.5 grams to about 7 grams, about 0.5 grams to about 8 grams, about 0.5 grams to about 9 grams, about 1 grams to about 2 grams, about 1 grams to about 3 grams, about 1 grams to about 4 grams, about 1 grams to about 5 grams, about 1 grams to about 6 grams, about 1 grams to about 7 grams, about 1 grams to about 8 grams, about 1 grams to about 9 grams, about 1 grams to about 10 grams, about 2 grams to about 3 grams, about 2 grams to about 4 grams, about 2 grams to about 5 grams, about 2 grams to about 6 grams, about 2 grams to about 7 grams, about 2 grams to about 8 grams, about 2 grams to about 9 grams, about 2 grams to about 10 grams, about 3 grams to about 4 grams, about 3 grams to about 5 grams, about 3 grams to about 6 grams, about 3 grams to about 7 grams, about 3 grams to about 8 grams, about 3 grams to about 9 grams, about 3 grams to about 10 grams, about 4 grams to about 5 grams, about 4 grams to about 6 grams, about 4 grams to about 7 grams, about 4 grams to about 8 grams, about 4 grams to about 9 grams, about 4 grams to about 10 grams, about 5 grams to about 6 grams, about 5 grams to about 7 grams, about 5 grams to about 8 grams, about 5 grams to about 9 grams, about 5 grams to about 10 grams, about 6 grams to about 7 grams, about 6 grams to about 8 grams, about 6 grams to about 9 grams, about 6 grams to about 10 grams, about 7 grams to about 8 grams, about 7 grams to about 9 grams, about 7 grams to about 10 grams, about 8 grams to about 9 grams, about 8 grams to about 10 grams, about 9 grams to about 10 grams, about 9 grams to about 10 grams, about 8 grams to about 10 grams, about 7 grams to about 10 grams, about 6 grams to about 10 grams, about 5 grams to about 10 grams, about 4 grams to about 10 grams, about 3 grams to about 10 grams, about 2 grams to about 10 grams, or about 1 gram to about 10 grams of each of the two or more fibers. In some embodiments, the kit includes include dosage forms (e.g., packets, gummies, tablets, and capsules) containing about 1 gram, 1.5 grams, about 1.8 grams, about 2 grams, about 2.4 grams, about 2.5 grams, about 3 grams, about 3.5 grams, about 4 grams, about 4.5 grams, about 5 grams, about 5.5 grams, about 6 grams, about 6.5 grams, about 7 grams, about 7.5 grams, about 8 grams, about 8.5 grams, about 9 grams, about 9.5 grams, or about 10 grams of each of the two or more fibers.

In some embodiments, the first fiber is any of the fibers described herein. In some embodiments, the second fiber is any of the fibers described herein, wherein the second fiber is different from the first fiber.

In some embodiments, the first fiber is psyllium and the second fiber is wheat dextrin. In some embodiments, the first fiber is psyllium and the second fiber is inulin. In some embodiments, the first fiber is wheat dextrin and the second fiber is inulin.

In some embodiments, the first fiber is beta-glucan and the second fiber is wheat dextrin. In some embodiments, the first fiber is beta-glucan and the second fiber is psyllium.

In some embodiments, the kit further includes a third fiber.

In some embodiments, the third fiber is present in an amount of about 1% to about 10% w/w of the dietary fiber combination. In some embodiments, the third fiber is present in an amount of about 1% to about 10% w/w of the composition.

In some embodiments, the ratio of the first fiber to third fiber is about 1:2 to about 10:1 first fiber:third fiber. For example, about 2:3, about 1:1, about 2:1, about 5:2, about 3:1, or about 3:2 first fiber:second fiber. In some embodiments, the ratio of the first fiber to third fiber is about 5:1 or about 10:1 first fiber:third fiber.

In some embodiments, the third fiber is an oligosaccharide is selected from the group consisting of: fructooligosaccharide, maltose, iso-maltose, a maltosyl-iso-malto-oligosaccharide, and a combination thereof. In some embodiments, the oligosaccharide is fructooligosaccharide. In some embodiments, the oligosaccharide is maltose.

In some embodiments, wherein the kit includes three fibers, the first fiber is beta-glucan, the second fiber is wheat dextrin, and the third fiber is psyllium.

The kit can further include instructions for the administration of the fibers (e.g., the first fiber and second fiber).

### Methods of Use

The compositions provided herein can be used in a variety of methods, including the treatment of a disease, disorder, or condition, and/or the improvement of a disease, disorder, or condition, for example, treating or improving one or more symptoms associated with the disease, disorder, or condition. Accordingly, provided herein are methods of treating a gastrointestinal disorder in a subject in need thereof that include administering to the subject a composition or dietary fiber combination, e.g., any of the compositions or dietary fiber combinations as described herein. Also provided herein are methods of improving a gastrointestinal disorder in a subject in need thereof that include administering to the subject a composition or dietary fiber combination, e.g., any of the compositions or dietary fiber combinations as described herein.

Also provided herein are methods for supporting a healthy digestive system in a subject in need thereof that include administering to the subject a composition or dietary fiber combination, e.g., any of the compositions or dietary fiber combinations as described herein. In some embodiments, provided herein are methods for improving or maintaining intestinal health in a subject in need thereof that include administering to the subject a composition or dietary fiber combination, e.g., any of the compositions or dietary fiber combinations as described herein.

In some embodiments, a subject in need thereof has one or more of: decreased bacterial diversity in the composition of their microbiome (e.g., a depletion of one or more bacterial strains, an overgrowth of one or more bacterial strains, or a combination thereof); an overgrowth of one or more populations of pathogens (e.g., a population of pathogenic bacteria); the presence of and/or overgrowth of a symbiotic organism able to cause disease when certain genetic and/or environmental conditions are present in the subject; and a shift in an ecological network that no longer provides a beneficial function to the subject and therefore no longer promotes health.

In some embodiments, provided herein are methods for altering the gut microbiome and/or metabolome in a favorable manner in a subject in need thereof that include administering to the subject a composition or dietary fiber combination, e.g., any of the compositions or dietary fiber combinations as described herein. In some embodiments, a method for altering the gut microbiome and/or metabolome in a favorable manner includes one or more of: increasing the diversity of the microbiome; decreasing the amount of bacteria that increase gut inflammation; increasing the amount of bacteria that decrease gut inflammation; altering the ratio of SCFA production; and altering the level of one or more bacterial metabolites. For example, in some embodiments, a method for altering the gut microbiome in a favorable manner includes restoring the ratio of butyrate, acetate, and propionate production (e.g., altering the ratio of butyrate, acetate, and propionate in a subject to the ratio found in a control population).

In some embodiments, provided herein are methods for increasing the diversity of the microbiome (e.g., the diversity of the bacteria, archaea, viruses, and/or fungi) and/or metabolome in the gastrointestinal tract of a subject in need thereof that include administering to the subject a composition or dietary fiber combination, e.g., any of the compositions or dietary fiber combinations described herein. In some embodiments, the levels of a gram-negative bacterial strain (e.g., a bacteria strain from the Acintobacter and Proteobacter families) and/or a gram-positive bacterial strain (e.g., a bacterial strain from the Bacterioidetes and Firmacutes families) can be restored. For example, a population of a gram-negative and/or gram-positive bacterial strain that is increased in a subject compared to the population of the same bacterial strain in a control individual or population can be decreased in the subject after administration of any of the compositions or dietary fiber combinations described herein. In some embodiments, the population of an increased bacterial strain can be decreased by at least 1%, at least 2%, at least 3%, at least 4%, at least 5%, at least 6%, at least 7%, at least 10%, at least 20%, at least 25%, at least 30%, at least 35%, at least 40%, or at least 50%, e.g., in a sample from the subject after administration of any of the compositions or dietary fiber combinations as described herein to the subject compared to before administration to the subject of any of the compositions or dietary fiber combinations described herein.

In some embodiments, a population of a gram-negative and/or gram-positive bacterial strain that is decreased in a subject compared to the population of the same bacterial strain in a control individual or population can be increased in the subject after administration of any of the compositions or dietary fiber combinations described herein. In some embodiments, the population of a decreased bacterial strain can be increased by at least 1%, at least 2%, at least 3%, at least 4%, at least 5%, at least 6%, at least 7%, at least 10%, at least 20%, at least 25%, at least 30%, at least 35%, at least 40%, or at least 50%, e.g., in a sample from the subject after administration to the subject of any of the compositions or dietary fiber combinations as described herein compared to before administration to the subject of any of the compositions or dietary fiber combinations described herein.

Some embodiments of any of the methods described herein include restoring the level of one or more bacterial metabolites (e.g., one or more of: lactate, an SCFA, a bile acid, a branched chain amino acid, tryptophan, TMAO, and an indole derivative) in the subject. Also provided herein are methods for restoring the level of one or more bacterial metabolites in a subject in need thereof, the method comprising administering to the subject a composition or dietary fiber combination, e.g., any of the compositions or dietary fiber combinations as described herein.

Non-limiting examples of bacterial metabolites include: lactate, SCFAs, bile acids, branched chain amino acids, tryptophan, trimethylamine N-oxide (TMAO), and indole derivatives. See, e.g., Agus et al. Gut. 2021; 70(6):1174-1182, which is herein incorporated by reference in its entirety. In some embodiments, the level of one or more bacterial metabolites (e.g., one or more of: lactate, SCFA, bile acid, branched chain amino acid, tryptophan, TMAO, and an indole derivative) is restored in the gastrointestinal tract of the subject.

In some embodiments of any of the methods described herein, a sample from the subject can be used to determine the composition of the microbiome in the sample and/or the level of one or more bacterial metabolites in the sample. Samples from the subject can be fecal samples, blood samples, plasma samples, serum samples, breath samples, or biopsy samples. Reference samples can be fecal samples, blood samples, plasma samples, serum samples, or biopsy samples. Fecal samples can be used to determine, for example, the composition of the microbiome and/or the level of one or more bacterial metabolites in the gastrointestinal tract of the subject or a control population.

In some of any of the above embodiments, the bacterial composition (e.g., of the gut microbiome) in a sample from the subject can be determined. For example, the methods provided herein can include detecting the presence or absence of a bacterial strain or relative amount of the bacterial strain in a sample from the subject (e.g., compared to a control population). In some embodiments, the gut microbiome can be analyzed by 16S rRNA unit subtyping.

In some embodiments, the ratio of different bacterial strains in a subject can be restored to a ratio determined from a reference sample (e.g., a sample from a control individual or an average of the level of the bacterial metabolite from samples from a control population).

Some embodiments of any of the methods described herein include detecting the level of one or more bacterial metabolites (e.g., one or more of: lactate, an SCFA, a bile acid, a branched chain amino acid, tryptophan, TMAO, and an indole derivative) in a sample from the subject. In some embodiments, the level of the bacterial metabolite (e.g., lactate, an SCFA, a bile acid, a branched chain amino acid, tryptophan, TMAO, or an indole derivative) in the sample from the subject is restored in comparison to the same bacterial metabolite in a reference sample. For example, the level of the bacterial metabolite in a sample from the subject can be increased or decreased to the level of the bacterial metabolite in a reference sample (e.g., a sample from a control population).

A variety of methods can be used to determine the level of a bacterial metabolite in a sample (e.g., a sample from a subject or a reference sample). Non-limiting examples of such methods include nuclear magnetic resonance spectroscopy, gas chromatography mass spectrometry (GC-MS), direct-injection gas chromatography, and tandem mass spectrometry. See, e.g., Moon et al. Nutrients. 2021 Jun. 24; 13(7):2180; Zheng et al. Metabolomics. 2013 Aug. 1; 9(4):818-827; Zhao et al. Biomed Chromatogr. 2006 August; 20(8):674-82; and Shan et al. Am J Clin Nutr. 2017; 106(3):888-894; each of which is herein incorporated by reference in its entirety.

Some embodiments of any of the methods described herein include detecting the level of one or more bacterial metabolites (e.g., one or more of: lactate, an SCFA, a bile acid, a branched chain amino acid, tryptophan, TMAO, and an indole derivative) in one or more samples from the subject (e.g., a first sample, a second sample, a third sample, etc.). For example, the level of one or more bacterial metabolites (e.g., one or more of: lactate, an SCFA, a bile acid, a branched chain amino acid, tryptophan, TMAO, and an indole derivative) can be detected in a first sample from the subject (e.g., a sample from the subject obtained prior to administration of any of the compositions or dietary fiber combinations described herein) and then the level of the one or more bacterial metabolites can be detected in a second sample from the subject (e.g., a sample from the subject obtained after administration of any of the compositions or dietary fiber combinations described herein). In some embodiments, a second sample from a subject (e.g., a sample from the subject obtained after administration of any of the compositions or dietary fiber combinations described herein) can be obtained after the subject has been administered any of the compositions or dietary fiber combinations described herein for a period of time. For example, the a second sample from a subject can be obtained after the subject has been administered any of the compositions or dietary fiber combinations described herein daily, every other day, or every three days for about 2 days to about 1 month.

Also provided herein are methods for improving intestinal health in a subject in need thereof, the method comprising: (a) determining that a sample from the subject (e.g., a first sample and/or a sample from the subject obtained prior to administration of any of the compositions or dietary fiber combinations described herein) has an increased level of one or more bacterial metabolites (e.g., one or more of: lactate, an SCFA, a bile acid, a branched chain amino acid, tryptophan, TMAO, and an indole derivative) compared to the level of the same bacterial metabolite from a reference sample (e.g., a sample from a control individual or an average of the level of the bacterial metabolite from samples from a control population); and (b) administering any of the compositions or dietary fiber combinations described herein. For example, the level of an increased bacterial metabolite can be decreased by at least 1%, at least 2%, at least 3%, at least 4%, at least 5%, at least 6%, at least 7%, at least 10%, at least 20%, at least 25%, at least 30%, at least 35%, at least 40%, or at least 50%, e.g., in a sample from the subject after administration of any of the compositions or dietary fiber combinations described herein to the subject compared to before administration to the subject of any of the compositions or dietary fiber combinations described herein. In some embodiments, the level of one or more bacterial metabolites (e.g., one or more of: lactate, SCFA, bile acid, branched chain amino acid, tryptophan, TMAO, and indole derivative) is decreased in a second sample from the subject (e.g., in a sample from the subject obtained after administration of any of the compositions or dietary fiber combinations described herein compared to the level of the same bacterial metabolite in the first sample (e.g., in a sample from the subject obtained prior to administration of any of the compositions or dietary fiber combinations described herein).

Also provided herein are methods for improving intestinal health in a subject in need thereof, the method comprising: (a) determining that a sample from the subject (e.g., a first sample and/or a sample from the subject obtained prior to administration of any of the compositions or dietary fiber combinations described herein) has a decreased level of one or more bacterial metabolites selected from: lactate, an SCFA, a bile acid, a branched chain amino acid, tryptophan, TMAO, and an indole derivative compared to the level of the same bacterial metabolite from a reference sample (e.g., a sample from a control individual or an average of the level of the bacterial metabolite from samples from a control population); and (b) administering any of the compositions or dietary fiber combinations described herein. For example, the level of a decreased bacterial metabolite can be increased by at least 1%, at least 2%, at least 3%, at least 4%, at least 5%, at least 6%, at least 7%, at least 10%, at least 20%, at least 25%, at least 30%, at least 35%, at least 40%, or at least 50%, e.g., in a sample from the subject after administration of any of the compositions or dietary fiber combinations described herein to the subject compared to before administration to the subject of any of the compositions or dietary fiber combinations described herein. In some embodiments, the level of one or more bacterial metabolites (e.g., one or more of: lactate, an SCFA, a bile acid, a branched chain amino acid, tryptophan, TMAO, and an indole derivative) is increased in a second sample (e.g., in a sample from the subject obtained after administration of any of the compositions or dietary fiber combinations described herein) from the subject compared to the level of the same bacterial metabolite in the first sample (e.g., in a sample from the subject obtained prior to administration of any of the compositions or dietary fiber combinations described herein).

Also provided herein are methods for reducing inflammation in a subject in need thereof comprising administering to the subject a composition or dietary fiber combination, e.g., any of the compositions or dietary fiber combinations described herein.

In some embodiments of any of the methods described herein, the subject has a gastrointestinal disorder. Non-limiting examples of gastrointestinal disorder include a foregut disease, flatulence, intestinal gas, bloating, leaky gut syndrome, constipation, hemorrhoids, anal fissure, diarrhea, fecal incontinence, irritable bowel syndrome, inflammatory bowel disease (Crohn's disease and ulcerative colitis), microscopic colitis, C. difficile colitis, small intestinal bacterial overgrowth, certain metabolic syndromes such as glucose intolerance, prediabetes, diabetes, coronary artery disease, nonalcoholic fatty liver disease, steatohepatitis, and obesity. In some embodiments, the gastrointestinal disorder (e.g., colitis) is induced by one or more chemotherapeutic agents, by treatment with adoptive cell therapy (e.g., immunotherapy), or by one or more alloimmune diseases (such as acute or chronic graft-vs-host disease). Non-limiting examples of a foregut disease include GERD, Barrett's esophagus, esophageal cancer (e.g., squamous or adenocarcinoma esophageal cancer), esophageal motility disorders, eosinophilic esophagitis, achalasia, oral cancer, halitosis, achalasia, esophageal diverticula, and periodontal gum disease.

In some embodiments, assessing a gastrointestinal disorder and/or gut microbiome health may include determining erythrocyte sedimentation rate (ESR), measuring the amount of C-reactive protein (CRP) (e.g., conducting a CRP test), or performing a clinical assessment to assess the severity or improvement of symptoms such as abdominal pain, bloating, constipation, diarrhea, nausea, dyspepsia. In some embodiments, a symptom score for diarrhea or constipation can be measured. A clinical assessment and/or symptom score may include, for example, the Bristol stool scale and Rome criteria (see, e.g., Lacy and Patel. J. Clin. Med. 2017; 6(11): 99, which is incorporated by reference herein in its entirety).

In some embodiments, treating a gastrointestinal disorder in a subject includes reducing one or more symptoms in the subject. For example, reducing flatulence, intestinal gas, abdominal pain, bloating, constipation, diarrhea, nausea, and/or dyspepsia. In some embodiments, the method includes administering to the subject any of the compositions as described herein. In some embodiments, the method includes administering a therapeutically effective amount of any of the compositions as described herein to the subject.

In some embodiments, improving a gastrointestinal disorder in a subject includes reducing one or more symptoms in the subject. For example, reducing flatulence, intestinal gas, abdominal pain, bloating, constipation, diarrhea, nausea, and/or dyspepsia. In some embodiments, the method includes administering a therapeutically effective amount of any of the compositions as described herein to the subject. In some embodiments, improving a gastrointestinal disorder in a subject includes providing relief from one or more symptoms in the subject selected from the group consisting of: flatulence, intestinal gas, abdominal pain, bloating, constipation, diarrhea, nausea, and/or dyspepsia. In some embodiments, the method includes administering to the subject any of the compositions or dietary fiber combinations as described herein.

In some embodiments, improving a state of diarrhea in a subject can refer to, for example, decreasing bowel movements, decreasing sudden urgency to defecate, and/or increasing stool hardness. In some embodiments, improving a state of constipation can refer to, for example, increasing bowel movements and/or softening of the stool.

In some of any of the above embodiments, the ratio of SCFAs in a sample from the subject is about 78:12:10 to about 60:20:20 acetate:propionate:butyrate after administration of any of the compositions or dietary fiber combinations described herein. For example, the ratio of SCFAs in the sample from the subject can be restored to a ratio found in a control population. See, e.g., the ratios of SCFAs as described in den Besten et al. J Lipid Res. 2013; 54(9):2325-40 and/or Jones et al. Sci Rep. 2021; 11(1):13964. In some embodiments, the ratio of SCFAs in the sample from the subject is about 78:12:10 acetate:propionate:butyrate after administration of any of the compositions or dietary fiber combinations described herein. In some embodiments, the ratio of SCFAs in the sample from the subject is about 60:20:20 acetate:propionate:butyrate after administration of any of the compositions or dietary fiber combinations described herein. SCFAs can be quantified using a variety of methods including direct-injection gas chromatography. See, e.g., Zhao et al. Biomed Chromatogr. 2006 August; 20(8):674-82. In some embodiments, the sample is a fecal sample.

In some of any of the above embodiments, the level of trimethylamine N-oxide (TMAO) in a sample from the subject is about 0.8 to about 2.2 μmol/L after administration of any of the compositions described herein. For example, about 0.8, about 1, about 1.2, about 1.4, about 1.6, about 1.8, about 2, or about 2.2 μmol/L after administration of any of the compositions described herein. TMAO can be quantified using a variety of methods. See, e.g., Shan et al. Am J Clin Nutr. 2017; 106(3):888-894. In some embodiments, the sample is a plasma sample.

In some of any of the above embodiments, the level of the indole derivative in a sample from the subject is about 15-25 nM after administration of the composition of any of the compositions described herein. For example, about 15, about 16, about 17, about 18, about 19, about 20, about 21, about 22, about 23, about 24, or about 25 nM after administration of the composition of any of the compositions described herein. Indole derivatives can be quantified using a variety of methods including ultraperformance liquid chromatography coupled to tandem mass spectrometry. See, e.g., Shan et al. Nat Commun. 2020; 11(1):5881. In some embodiments, the indole derivative is imidazole propionate. In some embodiments, the sample is a serum sample.

In some of any of the above embodiments, the level of the lactate in a sample from the subject is about 1 to about 6 mmol/L after administration of any of the compositions described herein. In some embodiments, the level of the lactate in the sample from the subject is 6 mmol/L or less after administration of any of the compositions described herein. Lactate can be quantified using a variety of methods including a lactic acid enzymatic kit. See, e.g., Mayeur et al. PLoS One. 2013; 8(1): e54335. In some embodiments, the sample is a fecal sample.

Some embodiments of any of the methods described herein include administering a composition or dietary fiber combination, e.g., any of the compositions or dietary fiber combinations as described herein, daily, every other day, or every three days.

In some embodiments, improving a gastrointestinal disorder in a subject in need thereof includes improving (e.g., decreasing) blood glucose levels in the subject. For example, the blood glucose level in the subject can decrease by at least about 10 mg/dl, e.g., at least 20 mg/dl, 30 mg/dl, 40 mg/dl, 50 mg/dl, 60 mg/dl, 70 mg/dl, 80 mg/dl, 90 mg/dl, 100 mg/dl or more. In some embodiments, the gastrointestinal disorder is diabetes or prediabetes.

In some embodiments, the subject is administered about 40% to about 60% w/w of the composition or dietary fiber combination of a first fiber; and about 40% to about 60% w/w of the composition or dietary fiber combination of a second fiber. In some embodiments, the subject is administered about 45% to about 55% w/w of the composition or dietary fiber combination of a first fiber; and about 45% to about 55% w/w of the composition or dietary fiber combination of a second fiber. In some embodiments, the subject is administered about 50% w/w of the composition or dietary fiber combination of the first fiber. In some embodiments, the subject is administered about 50% w/w of the composition or dietary fiber combination of the second fiber.

In some embodiments, the subject is administered a first fiber and a second fiber at a ratio of about 1:4 to about 4:1 first fiber:second fiber. For example, about 1:3, about 2:5, about 1:2, about 2:3, about 1:1, about 2:1, about 5:2, about 3:1, or about 3:2 first fiber:second fiber.

In some embodiments, the subject is administered a dosage of a composition or dietary fiber combination described herein of from about 2 grams to about 15 grams by weight of the composition or dietary fiber combination. For example, about 2 grams to about 4 grams, about 2 grams to about 6 grams, about 2 grams to about 8 grams, about 2 grams to about 10 grams, about 2 grams to about 12 grams, about 2 grams to about 14 grams, about 4 grams to about 6 grams, about 4 grams to about 8 grams, about 4 grams to about 10 grams, about 4 grams to about 12 grams, about 4 grams to about 14 grams, about 4 grams to about 15 grams, about 6 grams to about 8 grams, about 6 grams to about 10 grams, about 6 grams to about 12 grams, about 6 grams to about 14 grams, about 6 grams to about 15 grams, about 8 grams to about 10 grams, about 8 grams to about 12 grams, about 8 grams to about 14 grams, about 8 grams to about 15 grams, about 10 grams to about 12 grams, about 10 grams to about 14 grams, about 10 grams to about 15 grams, about 11 grams to about 15 grams, about 12 grams to about 14 grams, about 12 grams to about 15 grams, about 13 grams to about 15 grams, or about 14 grams to about 15 grams by weight of the composition or dietary fiber combination. In some embodiments, the subject is administered a dosage of a composition or dietary fiber combination described herein of from about 1 gram to about 3 grams, about 1 gram to about 5 grams, about 1 gram to about 10 grams, about 2 grams to about 4 grams, about 2 grams to about 6 grams, about 3 grams to about 5 grams, about 3 grams to about 6 grams, about 4 grams to about 8 grams, about 4 grams to about 10 grams, about 6 grams to about 10 grams, about 6 grams to about 12 grams, about 8 grams to about 12 grams, or about 8 grams to about 14 grams by weight of the composition or dietary fiber combination. In some embodiments, the subject is administered a dosage of a composition or dietary fiber combination described herein of from about 2 grams, about 2.4 grams, about 2.5 grams, about 3 grams, about 3.5 grams, about 3.6 grams, about 4 grams, about 4.2 grams, about 4.5 grams, about 4.8 grams, about 5 grams, about 5.5 grams, about 6 grams, about 6.5 grams, about 7 grams, about 7.5 grams, about 8 grams, about 8.5 grams, about 9 grams, about 9.5 grams, about 10 grams, about 10.5 grams, about 11 grams, about 11.5 grams, about 12 grams, about 12.5 grams, about 13 grams, about 13.5 grams, about 14 grams, about 14.5 grams, or about 15 grams by weight of the composition or dietary fiber combination.

In some embodiments, the subject is administered a dosage of a composition or dietary fiber combination described herein of about 1 gram to about 6 grams of a first fiber; and about 1 gram to about 6 grams of a second fiber. In some embodiments, the subject is administered about 2 grams to about 4 grams of the first fiber; and about 2 grams to about 4 grams of the second fiber. In some embodiments, the subject is administered about 2 grams to about 3 grams of the first fiber; and about 2 grams to about 3 grams of the second fiber. In some embodiments, the subject is administered about 2 grams of the first fiber and about 2 grams of the second fiber. In some embodiments, the subject is administered about 4 grams of the first fiber and about 4 grams of the second fiber. In some embodiments, the subject is administered about 2 grams of the first fiber and about 4 grams of the second fiber.

The foregoing dosages can be administered on a daily basis (e.g., as a single dose per day; or as two or more divided doses per day; or as two or more doses; e.g., two doses per day or three doses per day) or non-daily basis (e.g., every other day, every two days, every three days, once weekly, twice weeks, once every two weeks, once a month). In certain embodiments, dosages can be administered for about 1 week, about 2 weeks, about 3 weeks, about 4 weeks, about 5 weeks, about 6 weeks, about 7 weeks, about 8 weeks, about 3 months, about 6 months, about 1 year, or beyond.

In some embodiments, the subject is administered the first fiber and the second fiber daily or every other day. In some embodiments, the subject is administered the first fiber and the second fiber daily.

In some embodiments, a subject is administered a first fiber and then subsequently or simultaneously is administered a second fiber. In some embodiments, the first fiber is present as a first composition and the second fiber is present as a second composition. In some embodiments, the first fiber and the second fiber are present in a composition together.

In some embodiments, the first fiber is any of the fibers described herein. In some embodiments, the second fiber is any of the fibers described herein, wherein the second fiber is different from the first fiber.

In some embodiments, the first fiber is psyllium and the second fiber is wheat dextrin. In some embodiments, the first fiber is psyllium and the second fiber is inulin. In some embodiments, the first fiber is wheat dextrin and the second fiber is inulin.

In some embodiments, the first fiber is beta-glucan and the second fiber is wheat dextrin. In some embodiments, the first fiber is beta-glucan and the second fiber is psyllium.

In some embodiments, the subject is further administered a third fiber.

In some embodiments, the subject is administered about 1% to about 10% w/w of the composition or dietary fiber combination of the third fiber.

In some embodiments, the subject is administered a first fiber and a third fiber at a ratio of about 1:2 to about 10:1 first fiber:third fiber. For example, about 2:3, about 1:1, about 2:1, about 5:2, about 3:1, or about 3:2 first fiber:second fiber. In some embodiments, the subject is administered a first fiber and a third fiber at a ratio of about 5:1 or about 10:1 first fiber:third fiber

In some embodiments, the third fiber is an oligosaccharide is selected from the group consisting of: fructooligosaccharide, maltose, iso-maltose, a maltosyl-iso-malto-oligosaccharide, and a combination thereof. In some embodiments, the oligosaccharide is fructooligosaccharide. In some embodiments, the oligosaccharide is maltose.

In some embodiments, wherein the subject is administered three fibers, the first fiber is beta-glucan, the second fiber is wheat dextrin, and the third fiber is psyllium.

In some embodiments, a subject is administered a first fiber and a second fiber as described herein and then subsequently or simultaneously is administered a third fiber. In some embodiments, the first fiber is present as a first composition, the second fiber is present as a second composition, and third fiber is present as a third composition. In some embodiments, the first fiber, the second fiber, and third fiber are present in a composition together.

### EXAMPLES

The invention will be described in greater detail by way of specific examples. The following examples are offered for illustrative purposes, and to exemplify the oral compositions and methods described herein and are not intended to limit the invention in any manner. Many variations will suggest themselves and are within the full-intended scope. Those of skill in the art will readily recognize a variety of non-critical parameters that can be changed or modified to yield essentially the same results.

### Example 1

Irritable bowel syndrome subjects, either diarrhea-predominant, constipation-predominant, or mixed-pattern, were administered psyllium in a dose of 2-3 grams, in combination with wheat dextrin in a dose of 2-3 grams, daily for a minimum of 2 weeks Subject information is included in Table 2.

TABLE 2
Subject Information

| Subject | Date of | | Weight | | | | Mixed |
|---|---|---|---|---|---|---|---|
| # | Birth | Ethnicity | (lbs.) | IBS | Diarrhea | Constip. | Pattern |
| 001 | Jul. 13, 1992 | Caucasian | 140 | yes | yes | no | no |
| 002 | Jun. 20, 1981 | Caucasian | 170 | yes | yes | no | no |
| 003 | Feb. 8, 1999 | Black | 135 | yes | no | yes | no |
| 004 | Jul. 2, 2001 | Caucasian | 125 | yes | no | no | yes |
| 005 | Jul. 27, 1969 | Caucasian | 190 | no | no | yes | no |
| 006 | Oct. 12, 1963 | Black | 205 | no | yes | no | no |
| 007 | May 1, 1975 | Caucasian | 210 | no | no | yes | no |
| 008 | Apr. 16, 1984 | Asian | 145 | no | no | no | yes |
| 009 | May 12, 1995 | Caucasian | 190 | no | no | no | yes |
| 010 | Mar. 12, 1979 | Caucasian | 135 | yes | no | yes | no |

Each subject's symptoms were evaluated using a 6 point scale with a score of 0-5 with 5 representing the worst symptoms (bloating, gas, abdominal pain, altered bowel habit) and 0 representing complete absence of symptoms. The score is a composite, well-being score of all symptoms taken together.

TABLE 3
Subject Scores

| Subject | Start of Fiber | Initial | 2 week | Current | Adverse |
|---|---|---|---|---|---|
| # | treatment | Score | score | score | Events |
| 001 | Jan. 5, 2021 | 5 | 1 | 2 | none |
| 002 | Feb. 10, 2021 | 4 | 2 | 1 | none |
| 003 | Mar. 14, 2021 | 3 | 3 | 2 | none |
| 004 | Mar. 17, 2021 | 3 | 1 | 4 | none |
| 005 | Apr. 1, 2021 | 5 | 2 | 1 | none |
| 006 | Apr. 11, 2021 | 4 | 1 | 1 | none |
| 007 | May 7, 2021 | 4 | 2 | 1 | none |
| 008 | May 19, 2021 | 4 | 2 | 2 | none |
| 009 | Jun. 11, 2021 | 3 | 1 | 2 | none |
| 010 | Jun. 22, 2021 | 5 | 1 | 1 | none |

### OTHER EMBODIMENTS

It is to be understood that while the invention has been described in conjunction with the detailed description thereof, the foregoing description is intended to illustrate and not limit the scope of the invention which is defined by the scope of the appended claims. Other aspects, advantages, and modification are within the scope of the following claims.

What is claimed is:

1. A composition comprising a first fiber present in an amount of about 20% to about 80% w/w of the composition and a second fiber present in an amount of about 20% to about 80% w/w of the composition.

2. The composition of claim 1, wherein the first fiber is present in an amount of about 35% to about 65% w/w of the composition.

3. The composition of any one of claims 1-2, wherein the first fiber is present in an amount of about 45% to about 55% w/w of the composition.

4. The composition of any one of claims 1-3, wherein the first fiber is present in an amount of about 50% w/w of the composition.

5. The composition of any one of claims 1-4, wherein the second fiber is present in an amount of about 35% to about 65% w/w of the composition.

6. The composition of any one of claims 1-5, wherein the second fiber is present in an amount of about 45% to about 55% w/w of the composition.

7. The composition of any one of claims 1-6, wherein the second fiber is present in an amount of about 50% w/w of the composition.

8. The composition of any one of claims 1-7, wherein the first fiber is present in an amount of about 1 grams to about 6 grams.

9. The composition of any one of claims 1-8, wherein the first fiber is present in an amount of about 2 grams to about 4 grams.

10. The composition of any one of claims 1-9, wherein the first fiber is present in an amount of about 2 grams to about 3 grams.

11. The composition of any one of claims 1-10, wherein the second fiber is present in an amount of about 1 grams to about 6 grams.

12. The composition of any one of claims 1-11, wherein the second fiber is present in an amount of about 2 grams to about 4 grams.

13. The composition of any one of claims 1-12, wherein the second fiber is present in an amount of about 2 grams.

14. The composition of any one of claims 1-13, wherein the ratio of the first fiber to the second fiber is about 2:3 to about 3:2.

15. The composition of any one of claims 1-14, wherein the ratio of the first fiber to the second fiber is about 1:1.

16. The composition of any one of claims 1-15, wherein the first fiber is selected from the group consisting of: psyllium, pectin, guar, a lignin, cellulose, hemicellulose, resistant starch, a resistant dextrin, inulin, a chitin, pectin, a beta-glucan, potato starch, corn starch, and an oligosaccharide.

17. The composition of any one of claims 1-16, wherein the second fiber is selected from the group consisting of: psyllium, pectin, guar, a lignin, cellulose, hemicellulose, a resistant starch, a resistant dextrin, inulin, a chitin, pectin, a beta-glucan, potato starch, cornstarch and an oligosaccharide, wherein the second fiber is different from the first fiber.

18. The composition of any one of claims 1-17, wherein the first fiber is psyllium.

19. The composition of claim 18, wherein the second fiber is selected from the group consisting of: wheat dextrin, inulin, potato starch, pectin, cornstarch and an oligosaccharide.

20. The composition of claim 18, wherein the second fiber is wheat dextrin.

21. The composition of claim 18, wherein the second fiber is inulin.

22. The composition of claim 18, wherein the second fiber is potato starch.

23. The composition of any one of claims 1-17, wherein the first fiber is wheat dextrin.

24. The composition of claim 23, wherein the second fiber is selected from the group consisting of: psyllium, inulin, potato starch, pectin, cornstarch and an oligosaccharide.

25. The composition of claim 23, wherein the second fiber is inulin.

26. The composition of claim 23, wherein the second fiber is potato starch.

27. The composition of any one of claims 1-17, wherein the first fiber is beta-glucan.

28. The composition of claim 27, wherein the second fiber is selected from the group consisting of: psyllium, inulin, potato starch, pectin, cornstarch, wheat dextrin, and an oligosaccharide.

29. The composition of claim 27, wherein the second fiber is wheat dextrin.

30. The composition of claim 27, wherein the second fiber is psyllium.

31. The composition of any one of claims 1-30, wherein the composition further comprises a third fiber.

32. The composition of claim 31, wherein the third fiber is an oligosaccharide.

33. The composition of claim 32, wherein the oligosaccharide is selected from the group consisting of: fructooligosaccharide (FOS), maltose, iso-maltose, a maltosyl-iso-malto-oligosaccharide, and a combination thereof.

34. The composition of claim 33, wherein the oligosaccharide is FOS.

35. The composition of any one of claims 31-34, wherein the third fiber is present in an amount of about 1% to about 10% w/w of the composition.

36. A composition comprising psyllium present in an amount of about 45% to about 55% w/w of the composition and wheat dextrin present in an amount of about 45% to about 55% w/w of the composition.

37. A composition comprising psyllium present in an amount of 45% to about 55% w/w of the composition and inulin present in an amount of 45% to about 55% w/w of the composition.

38. A composition comprising wheat dextrin present in an amount of 45% to about 55% w/w of the composition and inulin present in an amount of 45% to about 55% w/w of the composition.

39. The composition of claim 36 or 37, wherein the psyllium is present in an amount of about 50% w/w of the composition.

40. The composition of claim 36 or 38, wherein the wheat dextrin is present in an amount of about 50% w/w of the composition.

41. The composition of claim 37 or 38, wherein the inulin is present in an amount of about 50% w/w of the composition.

42. The composition of any one of claims 36-41, wherein the composition further comprises an oligosaccharide.

43. The composition of claim 42, wherein the oligosaccharide is selected from the group consisting of: fructooligosaccharide (FOS), maltose, iso-maltose, a maltosyl-iso-malto-oligosaccharide, and a combination thereof.

44. The composition of claim 43, wherein the oligosaccharide is FOS.

45. The composition of any one of claims 42-44, wherein the oligosaccharide is present in an amount of about 1% to about 10% w/w of the composition.

46. The composition of any one of claims 19, 20, 22, 36, 38, and 40, wherein the wheat dextrin comprises about 0 to about 20 ppm of gluten.

47. The composition of any one of claims 1-46, wherein the composition further comprises a preservative.

48. The composition claim 47, wherein the preservative comprises: a palmitate, a benzoate, benzoic acid, a propionate, a sorbate, sorbic acid, an erythorbate, a nitrite, ethylenediaminetetraacetic acid (EDTA), butylated hydroxyanisole (BHA), butylated hydroxytoluene (BHT), capryllic acid, dilauryl thiodipropionate, erythorbic acid, gum guaiac, methylparaben, a sulfite, a bisulfite, a metabisulfite, propyl gallatepy, propylparaben, stannous chloride, sulfur dioxide, thiodipropionic acid, a tocopherol, or a combination thereof.

49. The composition of any one of claims 1-48, wherein the composition further comprises a sweetener.

50. The composition of claim 49, wherein the sweetener comprises: an intense sweetener, a natural sugar, or a combination thereof.

51. The composition of claim 50, wherein the sweetener comprises: sorbitol, sucrose, a saccharin, a cyclamate, aspartame, sucralose, thaumatin, acesulfam K, xylose, ribose, glucose, mannose, galactose, fructose, dextrose, sucrose, maltose, starch or corn syrup, sorbitol, xylitol, mannitol, glycerin, or a combination thereof.

52. The composition of any one of claims 1-51, wherein the composition further comprises a flavoring agent.

53. The composition of claim 52, wherein the flavoring agent comprises: a natural flavoring agent, a natural fruit flavoring agent, an artificial flavoring agent, an artificial fruit flavoring agent, a flavor enhancer, or a combination thereof.

54. The composition of any one of claims 1-53, wherein the composition comprises about 0 to about 20 ppm of gluten.

55. The composition of any one of claims 1-54, wherein the composition is a solid composition.

56. The composition of any one of claims 1-55, wherein the composition is a powder, a tablet, or a gummy.

57. The composition of any one of claims 1-56, wherein the composition is homogenous.

58. A method for reducing inflammation in a subject in need thereof, the method comprising administering to the subject a composition of any one of claims 1-57.

59. A method for improving intestinal health in a subject in need thereof, the method comprising administering to the subject a composition of any one of claims 1-57.

60. A method for improving a gastrointestinal disease in a subject in need thereof, the method comprising administering to the subject a composition of any one of claims 1-57.

61. A method for improving diabetes or prediabetes in a subject in need thereof, the method comprising administering to the subject a composition of any one of claims 1-57.

62. A method for improving blood glucose levels in a subject in need thereof, the method comprising administering to the subject a composition of any one of claims 1-57.

63. A method for increasing the diversity of the microbiome and/or metabolome in the gastrointestinal tract of a subject in need thereof, the method comprising administering to the subject a composition of any one of claims 1-57.

64. The method of any one of claims 58-62, wherein the method comprises increasing the diversity of bacteria in the gastrointestinal tract of the subject.

65. The method of any one of claims 58-64, wherein the method comprises determining the bacterial composition in a sample from the subject.

66. The method of any one of claims 58-65, wherein the method comprises restoring the level of one or more bacterial metabolites selected from: lactate, an SCFA, a bile acid, a branched chain amino acid, tryptophan, TMAO, and an indole derivative in the subject.

67. A method for restoring the level of one or more bacterial metabolites in a subject in need thereof, the method comprising administering to the subject a composition of any one of claims 1-57 to the subject.

68. The method of claim 67, wherein the level of one or more bacterial metabolites selected from: lactate, SCFA, bile acid, branched chain amino acid, tryptophan, TMAO, and indole derivative is restored in the gastrointestinal tract of the subject.

69. The method of any one of claims 58-68, wherein the method comprises detecting the level of one or more of: lactate, an SCFA, a bile acid, a branched chain amino acid, tryptophan, TMAO, and an indole derivative in a sample from the subject.

70. The method of any one of claims 66-69, wherein the level of the lactate, SCFA, bile acid, branched chain amino acid, tryptophan, TMAO, or indole derivative in the sample is restored in comparison to the same bacterial metabolite in a reference sample.

71. The method of any one of claims 65-70, wherein the sample is obtained after administration of the composition of any one of claims 1-57.

72. The method of claim 70 or 71, wherein the reference sample is a sample from the subject obtained prior to the administration of the composition of any one of claims 1-57.

73. The method of any one of claims 65-72, wherein the sample is a fecal sample.

74. The method of any one of claims 65-73, wherein the reference sample is a fecal sample.

75. A method for improving intestinal health in a subject in need thereof, the method comprising:
(a) determining that a first sample from the subject has an increased level of one or more bacterial metabolites selected from: lactate, an SCFA, a bile acid, a branched chain amino acid, tryptophan, TMAO, and an indole derivative compared to the level of the same bacterial metabolite in a reference sample; and
(b) administering to the subject a composition of any one of claims 1-57.

76. The method of claim 75, wherein the gastrointestinal tract of the subject has an increased level of the one or more bacterial metabolites.

77. The method of any one of claims 66-76, wherein the level of one or more bacterial metabolites selected from: lactate, SCFA, bile acid, branched chain amino acid, tryptophan, TMAO, and indole derivative is restored in a second sample from the subject compared to the level of the same metabolite in the first sample from the subject, wherein the second sample is obtained after administration of the composition of any one of claims 1-57.

78. The method of any one of claims 66-77, wherein the level of one or more bacterial metabolites selected from: lactate, SCFA, bile acid, branched chain amino acid, tryptophan, TMAO, and indole derivative is decreased in a second sample from the subject compared to the level of the same metabolite in the first sample from the subject, wherein the second sample is obtained after administration of the composition of any one of claims 1-57.

79. A method for improving intestinal health in a subject in need thereof, the method comprising:
(a) determining that a sample from the subject has a decreased level of one or more bacterial metabolites selected from: lactate, an SCFA, a bile acid, a branched chain amino acid, tryptophan, TMAO, and an indole derivative compared to the level of the same bacterial metabolite in a reference sample; and
(b) administering to the subject a composition of any one of claims 1-57.

80. The method of claim 79, wherein the gastrointestinal tract of the subject has a decreased level of the one or more bacterial metabolites.

81. The method of any one of claims 79-80, wherein the level of one or more bacterial metabolites selected from: lactate, SCFA, bile acid, branched chain amino acid, tryptophan, TMAO, and indole derivative is restored in a second sample from the subject compared to the level of the same metabolite in the first sample from the subject, wherein the second sample is obtained after administration of the composition of any one of claims 1-57.

82. The method of any one of claims 79-81, wherein the level of one or more bacterial metabolites selected from: lactate, SCFA, bile acid, branched chain amino acid, tryptophan, TMAO, and indole derivative is increased in a second sample from the subject compared to the level of the same metabolite in the first sample from the subject, wherein the second sample is obtained after administration of the composition of any one of claims 1-57.

83. The method of any one of claims 75-82, wherein the method comprises detecting the level of one or more bacterial metabolites selected from: lactate, an SCFA, a bile acid, a branched chain amino acid, tryptophan, TMAO, and an indole derivative in a first sample from the subject.

84. The method of claim 78 or 82, wherein the method comprises detecting the level of one or more bacterial metabolites selected from: lactate, an SCFA, a bile acid, a branched chain amino acid, tryptophan, TMAO, and an indole derivative in a second sample from the subject.

85. The method of any one of claims 75-84, wherein the first sample is a fecal sample.

86. The method of any one of claims 77, 78 and 81-85, wherein the second sample is a fecal sample.

87. The method of any one of claims 58-86, wherein the ratio of SCFAs in the sample from the subject is about 78:12:10 to about 60:20:20 acetate:propionate:butyrate after administration of the composition of any one of claims 1-57.

88. The method of any one of claims 58-87, wherein the ratio of SCFAs in the sample from the subject is about 78:12:10 acetate:propionate:butyrate after administration of the composition of any one of claims 1-57.

89. The method of any one of claims 58-87, wherein the ratio of SCFAs in the sample from the subject is about 60:20:20 acetate:propionate:butyrate after administration of the composition of any one of claims 1-57.

90. The method of any one of claims 58-89, wherein the level of TMAO in the sample from the subject is about 0.8 to about 2.2 μmol/L after administration of the composition of any one of claims 1-57.

91. The method of any one of claims 58-90, wherein the level of TMAO in the sample from the subject is about 1.5 μmol/L after administration of the composition of any one of claims 1-57.

92. The method of claim 90 or 91, wherein the sample is a plasma sample.

93. The method of any one of claims 58-92, wherein the level of the indole derivative in the sample from the subject is about 15-25 nM after administration of the composition of any one of claims 1-57.

94. The method of any one of claims 58-92, wherein the level of the indole derivative in the sample from the subject is about 20 nM after administration of the composition of any one of claims 1-57.

95. The method of claim 93 or 94, wherein the sample is a serum sample.

96. The method of any one of claims 58-95, wherein the indole derivative is imidazole propionate.

97. The method of any one of claims 58-96, wherein the level of the lactate in the sample from the subject is about 1 to about 6 mmol/L after administration of the composition of any one of claims 1-51.

98. The method of any one of claims 58-97, wherein the level of the lactate in the sample from the subject is 6 mmol/L or less after administration of the composition of any one of claims 1-51.

99. The method of any one of claims 58-98, wherein the subject has a gastrointestinal disease selected from the group consisting of: a foregut disease, flatulence, intestinal gas, bloating, leaky gut syndrome, constipation, hemorrhoid, anal fissure, diarrhea, fecal incontinence, irritable bowel syndrome, inflammatory bowel disease, microscopic colitis, small intestinal bacterial overgrowth, glucose intolerance, prediabetes, diabetes, coronary artery disease, nonalcoholic fatty liver disease, steatohepatitis, and obesity.

100. The method of any one of claims 58-99, wherein the subject has a gastrointestinal disease selected from the group consisting of: a foregut disease, flatulence, intestinal gas, bloating, leaky gut syndrome, constipation, hemorrhoid, anal fissure, diarrhea, fecal incontinence, dyspepsia, irritable bowel syndrome, inflammatory bowel disease, microscopic colitis, small intestinal bacterial overgrowth, glucose intolerance, prediabetes, diabetes, coronary artery disease, nonalcoholic fatty liver disease, steatohepatitis, and obesity.

101. The method of claim 99 or 100, wherein the foregut disease is selected from the group consisting of: GERD, Barrett's esophagus, esophageal cancer, esophageal motility disorders, eosinophilic esophagitis, achalasia, oral cancer, halitosis, achalasia, esophageal diverticula, and periodontal gum disease.

102. The method of any one of claims 58-101, wherein the composition is administered to the subject daily.

103. The method of any one of claims 58-102, wherein the composition is administered to the subject for about one day to about one month.

104. The method of any one of claims 58-103, wherein about 2 to about 10 grams of the composition is administered the subject daily.

105. The method of any one of claims 58-104, wherein about 4 to about 8 grams of the composition is administered the subject daily.

106. The method of any one of claims 58-105, wherein about 4 to about 8 grams of the composition is administered the subject daily.

107. The method of any one of claims 58-106, wherein about 4 grams of the composition is administered the subject daily.

108. The method of any one of claims 58-107, wherein about 8 grams of the composition is administered the subject daily

109. The method of any one of claims 58-108, wherein the sample is a breath sample.
